(19) United States
(12) Patent Application Publication
(10) Pub. No.: US 2008/0175888 A1
(43) Pub. Date: Jul. 24, 2008

(54) Combination Therapy Comprising Actinidia and Steroids and Uses Thereof

(76) Inventors: Julie Lindemann, Moraga, CA (US); Nancy Ellen Fogg-Johnson, Villanova, CA (US); Patti Cronin, Moraga, CA (US); Gil Belofsky, Longmont, CO (US); Dean P. Stull, Longmont, CO (US)

Correspondence Address: SHERIDAN ROSS PC, 1560 BROADWAY, SUITE 1200, DENVER, CO, 80202, US

(21) Appl. No.: 12/015,193

(22) Filed: Jan. 16, 2008

Related U.S. Application Data
(63) Continuation-in-part of application No. 11/817,216.
(60) Provisional application No. 60/885,210, filed on Jan. 16, 2007.
(60) Provisional application No. 60/656,838, filed on Feb. 25, 2005.
(60) Provisional application No. 60/656,839, filed on Feb. 25, 2005.

(51) Int. Cl. A61K 9/00; A61K 36/00; A61K 9/46; A61P 37/02; A61K 9/14

(52) U.S. Cl. 424/442; 424/777; 424/466; 424/489

(57) ABSTRACT

Disclosed is a combination of an Actinidia preparation and one or more steroids, and the use of such a combination to prevent and/or treat allergic and non-allergic inflammatory conditions or diseases, to alleviate at least one symptom of such conditions or diseases, and/or to regulate an immune response in a mammal.

### CROSS-REFERENCE TO RELATED APPLICATIONS

This application claims the benefit of priority under 35 U.S.C. § 119(e) from U.S. Provisional Application Ser. No. 60/885,210, filed Jan. 16, 2007. This application is also a continuation-in-part of U.S. application Ser. No. 11/817,216, filed Aug. 27, 2007, which is a national stage application under 35 U.S.C. § 371 of PCT Application No. PCT/US2006/006437, filed Feb. 24, 2006, which claims the benefit of priority under 35 U.S.C. § 119(e) to each of: U.S. Provisional Application Ser. No. 60/656,838, filed Feb. 25, 2005 and U.S. Provisional Application Ser. No. 60/656,839, filed Feb. 25, 2005. Each of U.S. Provisional Application Ser. No. 60/885,210, PCT Application No. PCT/US2006/006437, U.S. Provisional Application Ser. No. 60/656,838, and U.S. Provisional Application Ser. No. 60/656,839, is incorporated herein by reference in its entirety.

### FIELD OF THE INVENTION

The present invention relates to a combination of an Actinidia preparation and one or more steroids, including steroid-based compounds or compositions, and the use of such combination to prevent and/or treat allergic and non-allergic inflammatory conditions or diseases, to reduce at least one symptom of such conditions or diseases, or to regulate an immune response in a mammal.

### BACKGROUND OF THE INVENTION

Diseases involving inflammation are characterized by the influx of certain cell types and mediators, the presence of which can lead to tissue damage and sometimes death. Diseases involving inflammation are particularly harmful when they afflict certain organs and systems, such as the respiratory system, which can result in obstructed breathing, hypoxemia, hypercapnia and lung tissue damage, or in the skin, which can result in pruritis (itching), skin lesions, swelling, and scaling, or in joints, which can result in erosion and destruction of cartilage, collagen and bone, or in the cardiovascular system, which can result in myocardial infarction, or in the central nervous system, which can contribute to decreased cognition and Alzheimer disease.

Allergic (atopic) diseases are mediated in part by immunoglobulin E (IgE), while the type-2 T helper (Th2) cells, mast cells and eosinophils have also been shown to play important roles in the disease process (Maggi E., Immunotechnology, 3:233-244, 1998; Pawankar R., Curr. Opin. Allergy Clin. Immunol., 1:3-6, 2001; Vercelli D., Clin. Allergy Immunol., 16:179-196, 2002). Cytokines produced by the T lymphocyte subset known as “Th2 cells” include IL-4, IL-5, IL-10 and IL-13, and cytokines produced by the T lymphocyte subset known as “Th1 cells” include IFN-γ and IL-12, and have been reported to negatively regulate the Th2 pathways.

Atopic dermatitis (AD) is a chronic and relapsing inflammatory skin disease characterized by pruritic and eczematous skin lesions, along with elevated IgE levels. The incidence of AD appears to be increasing worldwide in infants and children. The skin lesions of AD patients are characterized by the infiltration of inflammatory cells including T lymphocytes, monocytes/macrophages, eosinophils and mast cells. These cells are involved in the pathogenesis and the development of AD through the release of various cytokines and chemokines such as IL-4, IL-5, IL-10, IL-13, eotaxin, and TARC. Among many cell types, Th2 cells producing IL-4, IL-5, IL-10, and IL-13 play a critical role in the initiating phase of the disease progression (Leung, 1997, Clin Exp Immunol 107(suppl. 1):25-30).

AD is also the second most common allergy in dogs (10% of canine population) (Scott et al., Small Animal Dermatology 5^{th} Ed, W B Saunders, 1995: 500-18). In canines, AD presents with pruritus and skin lesions that typically affect face, feet, ears and can easily become generalized. Typically, AD in dogs tends to worsen with age. Affected animals suffer from recurrent skin and ear infections that greatly decrease the quality of life of these patients.

Despite the fact that AD is a very common disease, the understanding of the pathogenesis of this disease and the therapeutic options available for affected patients are limited. Systemic treatments like glucocorticoids and cyclosporine may be quite effective in dogs (Olivry et al., Vet. Dermatol 2000; 11: 47) as well as humans, but have the potential for adverse effects (Ryffel et al., 1983, Arch. Toxicol 1983; 53: 107-41). Glucocorticoids are reasonably priced but tend to be less effective with chronic use (Scott et al., supra) and oral cyclosporine may be cost-prohibitive in large companion animals, such as large breed dogs. Antihistamines may be used, but the success rate is often unsatisfactory (Scott et al., supra). Hyposensitization is used in cases with a long season; however, clinical improvement is usually not seen for the first 6 to 9 months of therapy. Finally, all the currently available topical agents provide only partial and temporary relief. Thus, the identification of a safe and effective treatment to decrease the signs and symptoms of AD, particularly in humans and canines, would be of tremendous benefit.

Asthma is another significant atopic disease, affecting the respiratory system, which impacts millions of people worldwide. Asthma is typically characterized by periodic airflow limitation and/or hyperresponsiveness to various stimuli which results in excessive airways narrowing. Other characteristics can include inflammation of airways, eosinophilia and airway fibrosis. Heightened airway responsiveness is thought to result from a complex inflammatory cascade involving several cell types, including T lymphocytes and eosinophils. In allergic asthma, Th2 cytokines predominate over Th1 cytokines.

The hypothesis that reducing serum IgE levels could improve allergic symptoms has been demonstrated by clinical trials using chimeric anti-IgE antibody (CGP-51901) and recombinant humanized monoclonal antibody (rhuMAB-E25) (Fahy et al., Am. J. Respir. Crit. Care. Med., 155:1828-1834, 1997). Diacyl benzimidazole analogs and bacterial polysaccharides that inhibit IgE synthesis and secretion have been described in U.S. Pat. No. 6,369,091 and U.S. Patent Publication No. 20020041885, respectively.

Inflammation also plays a central role in osteoarthritis. In arthritis, the metabolic process of remodeling of bone and cartilage is altered, such that there is an imbalance between tissue breakdown and tissue repair. Medical evaluation has established that one underlying contributory cause is the altered metabolism of dietary fat, particularly arachidonic acid. In contrast to normal cartilage, OA cartilage spontaneously produces significant quantities of PGE217 and an array of cytokines, including IL-1β, IL-8, and IL-6 as well as other inflammatory mediators such as LTB₄ and lipoxins. In arthritis, arachidonic acid (AA) metabolism produces excess leukotrienes, and these in turn increase the expression/activity of matrix metalloproteinases (MMP), enzymes that degrade the protein matrix of cartilage (Martel-Pelletier et al., 2004). In addition, in ways that are not entirely understood, the altered fatty acid metabolism contributes to the over-production of IL-1β and TNFα, inflammatory cytokines that incite joint pain and inflammation

Inflammation also plays a central role in cardiovascular disease, and chronic inflammation and immune activation have been linked to enhanced risk for atherosclerosis. There is an association between common allergic diseases (e.g., allergic rhinitis and asthma) and 5-year development and progression of carotid atherosclerosis and high intimia-media thickness in carotid and femoral arteries. Patients with allergic disorders are at a significantly increased risk for high intimia-media thickness and for atherosclerosis development and progression. Enhanced atherosclerosis was observed among subjects with common allergic diseases and key components of allergies, such as leukotrienes or mast cells, are active in human atherogenesis. Accordingly, immune system-mediated and chronic inflammatory disorders are associated with enhanced risk for atherosclerosis.

Korea Patent Application No. 92-11752 disclosed an anti-inflammatory, anti-allergic and anti-rheumatic drug comprising biflavonoid such as 4′-O-methyl ochnaflavone isolated from Lonicera japonica, which shows efficacy in the treatment of various symptoms associated with allergy or inflammation. Korea Patent Registration No. 100744 disclosed an anti-inflammatory, anti-allergic and anti-rheumatic drug comprising several biflavonoid compounds isolated from the leaves of Ginko biloba. Several Oriental medicinal recipes comprising Siegesbeckia glabrescens have been reported to have IgE-reducing activity (Kim et al., Phytother. Res., 15:572-576, 2001). Furthermore, many medicinal herbs have been found to be rich sources of histamine release inhibitors or anti-inflammatory compounds.

U.S. Patent Publication No. 2004/0037909 by Kim et al., disclosed that extracts, including total water soluble extract and ethyl acetate extract, of hardy kiwifruit (Actinidia arguta, Actinidia polygama, Actinidia kolomikta) increase serum levels of Th1 cytokines and IgG2a, reduce serum levels of Th2 cytokines and IgE, inhibit histamine release from mast cells, and suppress allergic inflammatory reactions, including in an allergen-sensitized murine model of allergic inflammation and airway hyperresponsiveness, as well as in a rat paw edema assay (see U.S. Patent Publication No. 2004/0037909, supra).

Other conventional drugs for the treatment of allergic disorders include anti-histamines, steroidal or non-steroidal anti-inflammatory drugs and leukotriene antagonists. These agents, however, have the potential of serious side effect, including, but not limited to, increased susceptibility to infection, liver toxicity, drug-induced lung disease, and bone marrow suppression. Thus, such drugs have been limited in their clinical use for the treatment of inflammation, and particularly allergic inflammation. The use of anti-inflammatory and symptomatic relief agents is a serious problem because of their side effects or their failure to attack the underlying cause of an inflammatory response.

There is a continuing requirement for less harmful and more effective agents for treating inflammation. Thus, there remains a need for new products with lower side effect profiles, less toxicity and more specificity for the underlying cause of the inflammation.

### SUMMARY OF THE INVENTION

One embodiment of the invention relates to a method to regulate an immune response in a mammal. The method includes the step of administering at least one hardy kiwifruit preparation and at least one steroid to the mammal in an amount sufficient to regulate an immune response in the mammal.

Another embodiment of the invention relates to a method to reduce at least one symptom of inflammation in a mammal. The method includes the step of administering a hardy kiwifruit preparation and at least one steroid to the mammal in an amount sufficient to reduce said symptom of inflammation. The symptom of inflammation can include, but is not limited to, itching, redness, oozing and crusting, thickening, hair loss, and swelling of the skin.

In one aspect of either of the above-embodiments of the invention, the hardy kiwifruit can include: Actinidia arguta, Actinidia kolomikta and Actinidia polygama, with Actinidia arguta being one preferred embodiment.

In one aspect of either of the above-embodiments of the invention, the hardy kiwifruit preparation is an extract of the hardy kiwifruit. For example, the hardy kiwifruit preparation is produced by extraction in distilled water or extraction in a non-polar solvent. In one aspect, such preparation is produced by chromatographic purification of an aqueous extract. In any of the preparations, chromatographic purification may involve the use of a reverse phase chromatographic media, or a normal phase chromatographic media.

In one aspect of any of the embodiments of the invention, the steroid comprises a naturally occurring steroid. In another aspect, the steroid comprises a synthetic steroid. In another aspect, the steroid is selected from: animal steroids, plant steroids and fungal steroids. In one aspect, the steroid comprises a corticosteroid.

In one aspect of any of the embodiments of the invention, the mammal has or is at risk of developing a condition in which enhancement of a Th1 response and/or suppression of a Th2 response is desirable. In one aspect, the mammal has or is at risk of developing an allergic disease or non-allergic inflammatory disease. One exemplary allergic disease is a disease regulated by leukotrienes. In one aspect, the allergic disease is selected from: atopic dermatitis, asthma, food allergy, allergic rhinitis, and chronic urticaria.

In one aspect of any of the embodiments of the invention, the step of administering comprises administering one or both of the hardy kiwifruit preparation and the steroid with a carrier, adjuvant, or excipient to the mammal.

In one aspect of any of the embodiments of the invention, the step of administering comprises providing one or both of the hardy kiwifruit preparation and the steroid to the mammal as a tablet, a powder, an effervescent tablet, an effervescent powder, a capsule, a liquid, a suspension, a granule or a syrup.

In one aspect of any of the embodiments of the invention, the step of administering comprises providing one or both of the hardy kiwifruit preparation and the steroid to the mammal in one or more health foods.

In one aspect of any of the embodiments of the invention, the step of administering comprises administering one or both of the hardy kiwifruit preparation and the steroid as a topical composition.

In one aspect of any of the embodiments of the invention, the step of administering comprises providing one or both of the hardy kiwifruit preparation and the steroid to the mammal in a feed or feed ingredient.

In one aspect of any of the embodiments of the invention, the hardy kiwifruit preparation is formulated in the same composition with the steroid.

In another aspect of any of the embodiments of the invention, the hardy kiwifruit preparation is formulated in a different composition than the steroid, but administered contemporaneously with the steroid.

In one aspect of any of the embodiments of the invention, following a period of administration of the hardy kiwifruit preparation and the steroid, the mammal is administered with the hardy kiwifruit preparation in the absence of the steroid. For example, the period of administration of the hardy kiwifruit preparation and the steroid to a mammal prior to administration of the hardy kiwifruit preparation alone can be any increment of time from one day to several years. The period of administration of the hardy kiwifruit preparation to a mammal alone following the administration of the hardy kiwifruit preparation and the steroid can be any increment of time, from one day up to several years.

Yet another embodiment of the invention relates to a composition for regulating an immune response in a mammal or for reducing at least one symptom of inflammation in the mammal. The composition includes at least one hardy kiwifruit preparation and at least one steroid. In one aspect, the composition is selected from: a pharmaceutical composition, a health food, a food ingredient, an animal feed, and a cosmetic composition.

In one aspect of this embodiment, the hardy kiwifruit is selected from: Actinidia arguta, Actinidia kolomikta and Actinidia polygama.

In another aspect of this embodiment, the hardy kiwifruit preparation is an extract of the hardy kiwifruit. In one aspect, the hardy kiwifruit preparation is produced by extraction in distilled water or by extraction in a non-polar solvent. In one aspect, the hardy kiwifruit preparation is produced by chromatographic purification of an aqueous extract. In one aspect, the chromatographic purification involves the use of a reverse phase chromatographic media. In one aspect, the chromatographic purification involves the use of a normal phase chromatographic media.

In another aspect of this embodiment, the steroid comprises a naturally occurring steroid. In one aspect, the steroid comprises a synthetic steroid. In another aspect, the steroid comprises a steroid selected from the group consisting of: animal steroids, plant steroids and fungal steroids. In one aspect, the steroid comprises a corticosteroid.

In one aspect of this embodiment, the composition is formulated for oral administration. In one aspect, the composition is formulated for topical administration.

Another embodiment of the invention relates to an animal feed product, comprising any of the compositions described above and elsewhere herein, which include a hardy kiwifruit composition and a steroid composition. In one aspect, the animal feed product is selected from: a pet food, a livestock food, a pet treat, a pet chew, and a supplement.

### BRIEF DESCRIPTION OF THE FIGURES

FIG. 1 is a schematic of the process used in order to produce larger amounts of PG102T; this frozen or otherwise dried kiwifruit concentrate is also referred to as FD001 (FG refers to food grade carrier).

FIG. 2 shows the effect of three doses (0.25, 1.0, and 10 mg/mL) of FD001 (PG102T) on the relative degree of production of the cytokines IL-4, IL-5, IL-10, IL-13, and IFN-γ by OVA-stimulated mouse splenocytes following 3 days exposure in vitro. Cytokine levels were measure by ELISA. Each point represents the average of data from splenocytes of ten individual mice.

FIG. 3 shows the effect of three doses (0.25, 1.0, and 10 mg/mL) of an ethyl acetate (EtOAc) extract of FD001 (PG102T) on the relative degree of production of the cytokines IL-4, IL-5, IL-10, IL-13, and IFN-γ by OVA-stimulated mouse splenocytes following 3 days exposure in vitro. Cytokine levels were measure by ELISA. Each point represents the average of data from splenocytes of ten individual mice.

FIG. 4 shows the effect of three doses (0.25, 1.0, and 10 mg/mL) of an A. arguta fruit juice concentrate on the relative degree of production of the cytokines IL-4, IL-5, IL-10, IL-13, and IFN-γ by OVA-stimulated mouse splenocytes following 3 days exposure in vitro. Cytokine levels were measure by ELISA. Each point represents the average of data from splenocytes of ten individual mice.

FIGS. 5A and 5B indicate the activity of three doses of known immunosuppressant compounds on the relative degree of production of IL-13, and IFN-γ by OVA-stimulated mouse splenocytes following 3 days exposure in vitro. Cyclosporin was tested at 0.0083, 0.083, and 4.15 μM and dexamethasone was tested at 0.01, 0.1, and 1 μM (FIG. 5A) with each point representing the average of data from splenocytes of ten individual mice. Quercetin was tested at 1.0, 10, and 25 μM with each point representing the average of data from splenocytes of two individual mice (FIG. 5B). Cytokine levels were measure by ELISA.

FIGS. 6A and 6B show the effect of three doses (1.0, 3.0, and 10 mg/mL) of FD001, an ethyl acetate (EtOAc) extract of FD001, and the aqueous remainder from this process on the relative degree of production of IL-13 (FIG. 6A) and IFN-γ (FIG. 6B) by OVA-stimulated mouse splenocytes following 3 days exposure in vitro. Cytokine levels were measure by ELISA. Each point represents the average of data from splenocytes of eight individual mice.

FIGS. 7A and 7B show the effect of three doses (1.0, 3.0, and 10 mg/mL) of FD001 and a powdered form of FD001 (created for use in capsules) on the relative degree of production of IL-13 (FIG. 7A) and IFN-γ (FIG. 7B) by OVA-stimulated mouse splenocytes following 3 days exposure in vitro. Cytokine levels were measure by ELISA. Each point represents the average of data from splenocytes of eight individual mice.

FIGS. 8A and 8B show the effect of various preparations of A. arguta on the relative degree of production of IL-13 (FIG. 8A) and IFN-γ (FIG. 8B) by OVA-stimulated mouse splenocytes following 3 days exposure in vitro. Three doses (1.0, 3.0, and 10 mg/mL) each of FD001, a fruit juice concentrate, an extract prepared by boiling of the fresh fruit in water, and a room temperature water extract of the fruit were tested. Cytokine levels were measure by ELISA. Each point represents the average of data from splenocytes of eight individual mice.

FIGS. 9A and 9B show the effect of preparations of various plant parts of A. arguta on the relative degree of production of IL-13 (FIG. 9A) and IFN-γ (FIG. 9B) by OVA-stimulated mouse splenocytes following 3 days exposure in vitro. Three doses (1.0, 3.0, and 10 mg/mL) each of a fruit juice concentrate, individual extracts prepared by boiling of the bark, root, or stem in H₂O, and FD001 were tested. Cytokine levels were measured by ELISA. Each point represents the average of data from splenocytes of eight individual mice.

FIGS. 10A-10C highlight the distributional shift that occurred between days 1 and 14 of a human clinical trial in which subjects responded positively to treatment for atopic dermatitis (AD) as measured by the Physician's Global Assessment (PGA) for AD (scoring criteria shown in FIG. 10C). Subjects were administered placebo or 600 mg of FD001 (PG102T) daily (FIGS. 10A and 10B, respectively) and were using topical steroid treatment concomitantly.

FIG. 11 shows the effect of various concentrated chromatographic fractions (cuts F1, F2, F3 and F6) taken from a C-18 reverse phase media on the relative degree of production of IL-13 (FIG. 9A) and IFN-γ (FIG. 9B) by OVA-stimulated mouse splenocytes following 3 days exposure in vitro. Various doses (0.1, 0.25, 0.5, 1.0, 3.0, and 5 mg/ml) of individual fractions were tested along with the control material SG05-0217A which is equivalent to FD001. Cytokine levels were measured by ELISA. Each point represents the average of data from splenocytes of eight individual mice. These samples demonstrated significant inhibition of cytokine production in this assay. All samples are equal or more potent then the standard sample of SG05-0217A. Samples GB-1-21-F2 and GB-1-21-F3 are significantly more potent.

### DETAILED DESCRIPTION OF THE INVENTION

The present invention relates to the present inventors' discovery that the combination of a hardy kiwifruit preparation (defined below) and steroids can surprisingly synergize to provide an enhanced alleviation of the symptoms of atopic dermatitis. Further, it was observed that after the steroid was withdrawn, alleviation of the symptoms of atopic dermatitis could be maintained without the steroid, thus the kiwifruit preparation performed as a monotherapy following the combination therapy. These results were observed in canines suffering from atopic dermatitis, and are believed, without being bound by theory, to be extendable to other mammalian patients (e.g., humans) with atopic dermatitis, as well as to other inflammation-mediated conditions or diseases, and particularly, atopic (allergic) conditions or diseases.

In addition, the present inventors have shown that administration of a powdered form of the water soluble extract of A. arguta as described herein to adult human patients suffering from atopic dermatitis of moderate severity, significantly reduced the physician's global rating of clinical signs. Surprisingly, significant reduction was attained in a specific clinical symptom assessed by the patient themselves (Redness) and trends of decreasing severity were noted in other clinical symptoms of the disease in those patients who concomitantly used a topical steroid, as compared to patients using the steroid but who did not receive the hardy kiwifruit extract. This improvement clearly improved the patients' quality of life.

Together, these results indicate that the combination of preparations of hardy kiwifruit as described herein with steroid-based therapy provides improved efficacy in the treatment of atopic disease as compared to the use of either the hardy kiwifruit or steroid therapy alone. The inventors' data show that hardy kiwifruit preparations can be used as adjunctive therapy with other therapeutic agents to treat atopic disease or other diseases associated with immune dysregulation, and after withdrawal of a steroid, the kiwifruit preparation can be used as a monotherapy. Such combination compositions and combination sequence administration protocols of the present invention can be used to enhance the efficacy of other pharmacological and nutritional therapies, particularly in patients with atopic conditions, as well as in patients with other inflammation-mediated conditions, and importantly, can reduce the need of the patient to rely exclusively on conventional therapies such as steroids, that can have serious side effects. Indeed, the ability to reduce the amount, length of time, and/or frequency of steroid use, while maintaining or increasing improvement in a symptom(s) of an atopic condition, provides a significant benefit to any patient with such condition.

Accordingly, in one embodiment, the present invention relates to a combination of a preparation of kiwifruit and a steroid, provided together in a composition for concurrent use or in separate compositions for concurrent or sequential use (although sequential use of one composition can be contemporaneous with the use of the other composition), or concurrent followed by or preceded by sequential use, to treat or prevent an atopic condition in a mammal. Compositions can include, but are not limited to, a pharmaceutical composition; a nutraceutical composition; a composition useful as or with a health food product, health food or health beverage (including medical foods), or food ingredient (including human and animal (including domestic pet) food ingredients); or a cosmetic composition. Compositions are described in more detail below.

The invention includes the use of any members of the family, Actinidiaceae, and particularly any members of the genus Actinidia, including, but not limited to, the common kiwi known as A. chinensis or A. deliciosa, and the hardy kiwifruit known as A. arguta, A. polygama, or A. kolomikta, to provide compositions of kiwifruit with immune regulatory activity. Preferably, the kiwi is a hardy kiwifruit. The term “kiwifruit” can be used herein to generically refer to any member of the genus Actinidia, and includes the members of the hardy kiwifruit as discussed above, as well as members of the common kiwi, also as discussed above.

More than 30 species belonging to Actinidiaceae have been reported. Among those, the fruit of A. chinensis or A. deliciosa have been named “kiwi” and are popular edible fruits. Reference herein to “common kiwi” or “common kiwifruit” is intended to refer to A. chinensis or A. deliciosa. According to the present invention, reference to “hardy kiwifruit” or “hardy kiwi” refers to any of A. arguta, A. polygama, and A. kolomikta, or another species of the Actinidia genus related thereto that has the bioactive properties of A. arguta, A. polygama, and A. kolomikta as initially described in U.S. Patent Publication No. 2004/0037909, and as further described in PCT Publication No. WO 2006/093793, or as further described herein, particularly with regard to the anti-allergy properties and the immune response/cytokine/leukotriene-regulatory properties of the fruit or preparations thereof. Reference generally to a “kiwifruit” or “kiwifruit preparation” intends to refer to any species of Actinidia, including common and hardy kiwifruit.

Actinidia arguta, Actinidia polygama, and Actinidia kolomikta belonging to Actinidiaceae, are naturally distributed in Siberia, the northern area of China, Japan, North and South Korea. A. arguta and other fruit of the same genus (e.g., A. polygama, and A. kolomikta) have been used as materials of Chinese medicine named as ‘mihudo’ to treat liver disease, gastrointestinal disease and urogenital lithiasis without toxicity (Seoul National University Natural Products Science, Tradi-Medi Data Base, dongbang media Co. Ltd. 1999). As discussed above, U.S. Patent Publication No. 2004/0037909, discloses the treatment and prevention of allergic disease and non-allergic inflammatory disease using various extracts of the hardy kiwifruit species of Actinidia.

Prior to the work of the present inventors, however, there have been no reports of an effect of the hardy kiwifruit compositions on the efficacy of established treatments for atopic disease or other conditions associated with dysregulation of the mammalian immune system (e.g., steroid therapy). The present inventors are believed to be the first to recognize that the combination of kiwifruit preparations and steroids provides an adjunctive and particularly, a synergistic, effect in the treatment of inflammatory conditions such as atopic dermatitis. Moreover, the present inventors are believed to be the first to discover that the use of the kiwifruit composition alone (as a monotherapy) directly after its use in combination with a steroid, can be an effective monotherapy for treatment of atopic or inflammatory conditions such as atopic dermatitis. This surprising discovery forms the basis of the present invention.

### Preparations, Formulations, Active Ingredients and Compositions of the Invention

It is an object of the present invention to provide a composition that comprises, consists essentially of, or consists of, a kiwifruit preparation and one or more steroids, including steroid-based compounds or compositions. Alternatively, a kiwifruit preparation is used together with and/or sequentially with a second composition comprising, consisting essentially of, or consisting of, one or more steroids, including steroid-based compounds or compositions (discussed below). Preferably, the steroid and hardy kiwifruit composition are used together in the same composition or are used contemporaneously. In one embodiment, periods of combined use can be followed or preceded by periods of monotherapy with the kiwifruit preparation alone. The compositions of the invention can be provided as a pharmaceutical composition, a nutraceutical composition, a dietary or nutritional supplement, a food additive or food ingredient or feed (including any pet food), a health food (including a beverage or a food material, and including medical food), or a cosmetic composition.

In one aspect, the composition comprises, consists essentially of, or consists of, the hardy kiwifruit or common kiwi described herein (i.e., including any part of the fruit, the whole fruit, the stem, the leaf, the bark, or the root, and including any preparation, concentrate or extract (including any crude extract, total water-soluble extract, or ethyl acetate extract) or concentrate thereof, including dried preparations, non-extracted but processed preparations, fresh fruit, fruit juice, or any extract, concentrate or chromatographic fraction thereof), in admixture with a steroid, including a steroid-based compound or composition, or provided as a composition to be used concurrently with or sequentially with a steroid, including a steroid-based compound or composition.

In the preparation of any composition or preparation of the invention or used in the invention, including any extract described herein, one may use any one or more parts of the hardy kiwifruit or other species of Actinidia, including, but not limited to, the fruit (also referred to as the “berries” or the “kiwiberries”), the leaf, the stem, the bark and the root thereof.

One extraction process used to produce a total water soluble extract and an ethyl acetate extract of hardy kiwifruit is described in U.S. Patent Publication No. 2004/0037909. Preparations of hardy kiwifruit produced by alternate methods of extraction, concentration or processing also produce compositions with immune regulatory activity, and particularly, the ability to suppress cytokine production in response to an antigen (e.g., an allergen). Such alternate preparations include, but are not limited to, fruit juice concentrate, fresh fruit concentrate, boiled fresh fruit preparations and preparations concentrated by chromatography techniques. Such preparations, in addition to those described in U.S. Patent Publication No. 2004/0037909, are useful in the present invention.

Extracts or other preparations of parts of the hardy kiwifruit plant other than the fruit itself have equivalent or superior immune regulatory activity as compared to the water soluble or ethyl acetate extracts of the fruit. For example, extracts of the stem, root and bark of the hardy kiwifruit plant are effective to suppress cytokine production by antigen-stimulated splenocytes from allergen-sensitized mice.

In addition, in any of the hardy kiwifruit preparations used in the invention, the process step of drying hardy kiwifruit is an important element to enhancing the bioactivity of the hardy kiwifruit. Accordingly, dried hardy kiwifruit that has not been extracted (e.g., dried slices of hardy kiwifruit) can have the bioactivity that was previously ascribed to extracts of hardy kiwifruit. Therefore, the present invention includes the use of any form of dried hardy kiwifruit, including extracts produced from previously dried hardy kiwifruit, as an agent or for the preparation of a composition for use in any of the methods of the invention, such as for regulating an immune response in a mammal or ameliorating at least one symptom of a disease or condition associated with inflammation, and particularly, allergic inflammation.

In another embodiment, preparations of common kiwifruit (e.g., A. chinensis or A. deliciosa) are useful in the present invention, including, but not limited to, preparations of any part of the fruit, the whole fruit, the stem, the leaf, the bark, or the root, and including any preparation or extract thereof, including dried preparations, non-extracted but processed preparations, fresh fruit, fruit juice, or any extract, concentrate, or fraction thereof, can have at least some of the biological properties that have been recognized for the hardy kiwifruit described herein.

General reference herein to an extract refers to a concentrated preparation of a substance (e.g., hardy kiwifruit) which is typically obtained by removing the active or desired constituents therefrom with a suitable solvent, and then evaporating some, all or nearly all the solvent and adjusting the residual mass or powder to a prescribed standard. The term “concentrate” refers to a form of substance which has had the majority of its base component, or solvent, removed. Accordingly, the term “extract” as disclosed herein may be, in some embodiments, used interchangeably with the term “concentrate”. Reference herein to a crude extract refers to, in one embodiment, an extract of hardy kiwifruit that is obtained by extracting a preparation of a hardy kiwifruit with water, lower alcohols (e.g., methanol, ethanol and the like), or the mixtures thereof, and preferably distilled water or 50-90% ethanol, and more preferably 70% ethanol. A non-polar solvent soluble extract therefrom can be obtained by further extracting the soluble extract with a non-polar solvent such as hexane, ethyl acetate or dichloromethane solvent. Specific procedures for production of a crude extract and evaluation of the same are described in U.S. Patent Publication No. 2004/0037909, supra, and all such procedures are incorporated herein by reference. Additional bioassays for tracking, evaluating or confirming the preferred biological activities of a hardy kiwifruit composition according to the present invention are set forth in the Examples herein, and include both in vitro and in vivo assays.

According to the present invention, reference to “FD001” refers generally to a total water-soluble extract (which may also be referred to herein as a concentrate) from a hardy kiwifruit described herein (e.g., A. arguta), prepared essentially as described in U.S. Patent Publication No. 2004/0037909, supra (e.g., see Example 1 of that publication), although FD001 typically refers to a large scale production of such an extract (the process steps are substantially similar, although adapted for large scale production). Such a total water-soluble extract is typically prepared from A. arguta, although it will be apparent to those of skill in the art that equivalent total water-soluble extracts can be prepared from other hardy kiwifruit, including, but not limited to, A. polygama and A. kolomikta. Other extracts can be prepared from hardy kiwifruit, including ethyl acetate fractions. Ethyl acetate fractions are disclosed in U.S. Patent Publication No. 2004/0037909, supra, and can be produced by successive solvent partition of a water-soluble extract described above with chloroform (optionally), ethyl acetate, and n-butanol using extraction methods conventionally known in the art.

In some embodiments of the invention, particularly when the fruit is dried as a step in the processing or preparation of an extract or concentrate or other preparation, extracts or concentrates or other preparations of the invention can be produced from any species of Actinidia. Accordingly, it is an object of the present invention to provide a composition that comprises such extracts of hardy kiwifruit or other species of Actinidia, if desired. Other additives, ingredients and components of the compositions, as well as the dosing and administration strategies described herein apply to this object of the invention as well.

In the preparation of any composition described herein, in addition to the extracts described herein, including the extracts or concentrates described specifically above, included in the invention is the use of whole fruits of hardy kiwifruit, or fruit preparations that are processed, but not extracted, including, but not limited to, fresh fruit, crushed fruit (dried or fresh), boiled fruit (dried or fresh), cooked fruit, dried fruit, pressed fruit, frozen fruit, and condensed fruit. Accordingly, it is an object of the present invention to provide a composition that comprises such whole fruits of hardy kiwifruit or fruit preparations that may be processed in some manner (e.g., dried, boiled, etc.), but that are not necessarily extracted. Therefore, in one embodiment, the present invention relates to preparations of hardy kiwifruit and/or common kiwifruit that have not been extracted. Any of such compositions described herein are intended for use in any method of the invention. Other additives, ingredients and components of the compositions, as well as the dosing and administration strategies described herein apply to this object of the invention as well.

Furthermore, in the preparation of any composition described herein, in addition to the extracts described herein, included in the invention is the use of the juice of a hardy kiwifruit or common kiwifruit, produced from any part of the hardy kiwifruit and by any suitable process. The juice can be used as produced directly from the fruit (i.e., not diluted or concentrated), the juice can be diluted, or it can be concentrated to form a fruit juice concentrate. Such concentration may not involve the use of an extraction solvent. For example, as described in the Examples, fresh kiwifruit can be run through a conventional juicer. The juicer may remove the skins from the fruit resulting in a mixture of seeds, pulp, and juice. This mixture can then be treated (e.g., by centrifugation or pressing) to remove the juice from the solids and, if desired, this juice can be concentrated (e.g., by evaporation, distillation, ultrafiltration, etc.) to provide a concentrated fruit juice (i.e., fruit juice concentrate). Such compositions are intended for use in any method of the invention. Other additives, ingredients and components of the compositions, as well as the dosing and administration strategies described herein apply to this object of the invention as well.

Furthermore, in the preparation of any composition described herein, as an alternative to the extracts described in U.S. Patent Publication No. 2004/0037909, included in the invention is the use of other products of processing of the hardy kiwifruit, including, but not limited to, a room temperature water extract of fruit, including dried fruit; a water extract of hardy kiwifruit performed in water having a temperature of less than room temperature; a water extract or other extract of the root, leaf, stem, or bark; or a water extract or concentrate or other extract of any of the fruit, leaf, stem or bark, that is not dried prior to extraction (e.g., extracted fresh fruit). The hardy kiwifruit can be extracted in any temperature water ranging from 0° C. to 80° C., including room temperature and cooler (e.g., from about 0° C. to about 25° C.).

According to the present invention, general reference to a “dried hardy kiwifruit” or a “dried kiwifruit” includes any form of a hardy kiwifruit or other kiwifruit (e.g., common kiwi) that has been dried by any process. Therefore, a dried kiwifruit includes any dried part of the kiwifruit (fruit, leaf, stem, root, etc.), and includes dried whole fruits, dried sliced fruit, dried crushed fruit, dried diced fruit, and dried condensed fruit, as well as any extracts of kiwifruit, wherein the material that is extracted is first dried prior to extraction. The extracts themselves need not be dried or processed further, although that is generally the most useful form for the extracts for formulation into compositions and for storage. Preferred methods of further concentration of the extracts for further use include, but are not limited to, evaporation, distillation, ultrafiltration, reverse osmosis, precipitation, adsorption to and elution from a stationary phase, chromatographic purification and extraction into alternative solvents. Preferred methods of drying the extracts or concentrates for further use include, but are not limited to, tray drying, spray drying and freeze drying, both with or without the use of drying aids or excipients such as maltodextrin, microcrystalline cellulose and starch. In one embodiment, a preferred dried kiwifruit for use in the present invention is a dried kiwifruit preparation that is not subsequently extracted (i.e., it is used in a composition or method of the invention without being extracted). In one aspect, the hardy kiwifruit is air dried between room temperature and 80° C. or vacuum dried between −170° C. to room temperature.

Therefore, the compositions and methods described herein apply to the use of any hardy kiwifruit (or common kiwi), including the use of any part of the fruit, stem, leaf, bark, root or other part of the hardy kiwifruit (or common kiwi), or any extract or concentrate or fraction thereof, any form of the whole fruit or processed but not extracted fruit, fruit juice, or any extract or concentrate or fraction thereof, and further including hardy kiwifruit (or common kiwi) plant parts (the plant parts including the fruit, stem, leaf, bark, and/or root) and plant part preparations that are prepared using a process that includes a step of drying.

According to the present invention, a composition is provided that includes one or more steroids, including steroid-based compounds or compositions, which represents an active ingredient in a composition of the invention. The term “steroid” generally refers to any steroid or steroid-based compound or steroid-based composition, many of which are described herein and/or are known in the art. Such a steroid composition can be the same composition as that comprising the hardy kiwifruit or common kiwi preparation discussed above, or can be a separate composition that is intended for use together with and/or sequentially with the hardy kiwifruit or common kiwi preparation.

According to the present invention, a steroid is defined as a terpenoid lipid characterized by a carbon skeleton with four fused rings. Different steroids vary in the functional groups attached to these rings. Hundreds of distinct steroids have been identified in plants, animals, and fungi. Steroids are derived either from the sterol lanosterol (animals and fungi) or the sterol cycloartenol (plants). Both sterols are derived from the cyclization of the triterpene squalene. Steroids include, but are not limited to, any naturally occurring or synthetic steroid including animal steroids, plant steroids and fungal steroids, and functional derivatives thereof. Animal steroids include, but are not limited to, insect steroids (e.g., Ecdysteroids such as ecdysterone) and vertebrate steroids. Vertebrate steroids include, but are not limited to, sex steroids (e.g., androgens, estrogens, progestagens), corticosteroids (e.g., glucocorticoids and mineralocorticoids), and anabolic steroids. Plant steroids include, but are not limited to, phytosterols and barssinosteroids. Fungal steroids include, but are not limited to ergosterols. In preferred embodiments of the invention, a steroid useful in a composition of the invention is any steroid with anti-inflammatory activity.

Corticosteroids are particularly preferred steroids for use in the present invention. Some preferred steroids and their derivatives for use in the present invention include, but are not limited to amcinonide, betamethasone, betamethasone dipropionate, betamethasone valerate, budesonide, clobetasol, clobetasol acetate, clobetasol butyrate, clobetasol 17-propionate, cortisone, deflazacort, desoximetasone, diflucortolone valerate, dexamethasone, dexamethasone sodium phosphate, desonide, furoate, fluocinonide, fluocinolone acetonide, halcinonide, hydrocortisone, hydrocortisone butyrate, hydrocortisone sodium succinate, hydrocortisone valerate, methyl prednisolone, mometasone, prednicarbate, prednisolone, triamcinolone, triamcinolone acetonide, and halobetasol proprionate.

One of skill in the art will appreciate that any steroid that is used in a mammal, and particularly, humans, companion animals, or livestock animals, can be useful in the present invention.

Steroids can be formulated for any type of administration, but are most often administered orally or topically. Steroids and steroid-based compounds or compositions can be naturally and/or synthetically derived, obtained, or produced.

The biological activity or biological action of any compound, preparation, or agent described herein, including a hardy kiwifruit preparation or steroid for use in the invention, refers to any function(s) exhibited or performed by the compound, preparation or agent that is ascribed to the naturally occurring form of the compound, preparation or agent as measured or observed in vivo (i.e., in a natural physiological environment wherein the compound, preparation or agent is used) or in vitro (i.e., under laboratory conditions). Modifications of a compound, preparation or agent, such as by changing the processing or preparation of the compound, preparation or agent or purification of the compound, preparation or agent, may result in compounds, preparations or agents having the same biological activity as the naturally occurring compound, preparation or agent, or in compounds, preparations or agents having decreased or increased biological activity as compared to the naturally occurring compound, preparation or agent.

As discussed above, a kiwifruit preparation, and preferably, a hardy kiwifruit preparation, is one of the active ingredients of the invention for use to selectively regulate Th1 and Th2 immune responses in a patient (i.e., in a mammal). Such a composition has a biological activity selected from at least one of the following activities: (a) reduces the number of IgE-producing B cells in a patient; (b) reduces the amount of IgE produced in a patient (e.g., in the serum or plasma); (c) decreases production and/or levels of at least one Th2 cytokine (e.g., IL-4, IL-5, IL-10); (d) increases the level of at least one Th1 cytokine (e.g., IL-12, IFN-γ); (e) decreases the level of expression of the transcription factor, GATA-3; (f) increases the level of expression of the transcription factor T-bet; (g) increases the level of expression of the transcription factor NFATc2; (h) increases the number of IgG2a-producing B cells in a patient; (i) increases the amount of IgG2a produced in a patient; (j) enhanced production or activity of Th1 T lymphocytes (e.g., CD4+, IFN-γ+), particularly at a site of inflammation; (k) decreases production or activity of Th2 T lymphocytes (e.g., CD4+, IL-4+), particularly at a site of inflammation; (l) reduces the number of IgG1-producing B cells in a patient; (m) reduces the amount of IgG1 produced in a patient; and/or (n) reduces the level of or production of at least one leukotriene in the patient.

The biological activity of a composition or combination of compositions of the invention can include regulation of an immune response in the mammal, and more particularly for regulation of a Th2 and/or a Th1 immune response in a mammal, and even more particularly, for enhancement of a Th1 response in a mammal and/or suppression of a Th2 response in a mammal. The active ingredients used in the composition or compositions of the invention are useful for the treatment, prevention, improvement, and/or alleviation of at least one symptom of a variety of conditions and diseases, including, but not limited to, allergic disease, non-allergic inflammatory disease, viral infection and cancer. Such symptoms, diseases and conditions are discussed in detail below.

According to the present invention, nutritional (nutraceutical) applications, and accordingly nutritional compositions, include any applications of the invention directed to the provision of nutrients and nutritional agents to maintain, stabilize, enhance, strengthen, or improve the health of an individual or the organic process by which an organism assimilates and uses food and liquids for functioning, growth and maintenance, and which includes nutraceutical applications. Therapeutic applications, and accordingly, therapeutic or pharmaceutical compositions, include any applications of the invention directed to prevention, treatment, management, healing, alleviation and/or cure of a disease or condition that is a deviation from the health of an individual, and can be referred to interchangeably as pharmaceutical applications. Other applications of the invention include, for example, cosmetic applications, which may or may not be considered to be therapeutic/pharmaceutical or nutritional in nature.

Accordingly, it is one object of the present invention to provide a nutritional, therapeutic (or pharmaceutical), or cosmetic composition, comprising a hardy kiwifruit preparation(s) described herein, formulated together with and/or provided sequentially with one or more steroid, including steroid-based compounds or compositions. Such compositions can be used for regulating an immune response in the mammal, and more particularly for regulating a Th2 and/or a Th1 immune response in a mammal, and even more particularly, for enhancing a Th1 response in a mammal and/or suppressing a Th2 response in a mammal. Such compositions are also useful for the treatment, prevention, improvement of, and/or alleviation of at least one symptom of a variety of conditions and diseases, including, but not limited to, allergic disease, non-allergic inflammatory disease, viral infection and cancer.

It is another object of the present invention to provide a health food or food ingredients (also sometimes referred to as food additives) comprising the hardy kiwifruit preparation(s) described herein, formulated together with and/or provided sequentially with one or more steroid, including steroid-based compounds or compositions. Such health foods or health food ingredients can be used for regulating an immune response in the mammal, and more particularly for regulating a Th2 and/or a Th1 immune response in a mammal, and even more particularly, for enhancing a Th1 response in a mammal and/or suppressing a Th2 response in a mammal. Such health food or health food ingredients are also useful for the treatment, prevention, improvement of, and/or alleviation of at least one symptom of a variety of conditions and diseases, including, but not limited to, allergic disease, non-allergic inflammatory disease, viral infection and cancer.

It is another object of the present invention to provide a medical food (including food and beverages) comprising the hardy kiwifruit preparation(s) described herein, formulated together with and/or provided sequentially with one or more steroids, including steroid-based compounds or compositions. Such medical foods can be used for regulating an immune response in the mammal, and more particularly for regulating a Th2 and/or a Th1 immune response in a mammal, and even more particularly, for enhancing a Th1 response in a mammal and/or suppressing a Th2 response in a mammal. Such medical foods are also useful for the treatment, prevention, improvement of, and/or alleviation of at least one symptom of a variety of conditions and diseases, including, but not limited to, allergic disease, non-allergic inflammatory disease, viral infection and cancer. According to the present invention and the FDA, a medical food is a food which is formulated to be consumed or administered under the supervision of a physician and intended for the specific dietary management of a disease for which distinctive nutritional requirements are established. Accordingly, as used herein, the terms “medical food” and “health food” can be used interchangeably in some circumstances. Specifically, a medical food is a subset of the category of health food, wherein the food, as discussed above, is formulated to be consumed or administered under the supervision of a physician and intended for the specific dietary management of a disease for which distinctive nutritional requirements are established.

It is still another object of the present invention to provide an animal feed or feed ingredient (sometimes also referred to as a feed or food additive) comprising the hardy kiwifruit preparation(s) described herein, formulated together with and/or provided sequentially with one or more steroids, including steroid-based compounds or compositions. Such animal feeds or feed ingredients can be used for regulating an immune response in the mammal, and more particularly for regulating a Th2 and/or a Th1 immune response in a mammal, and even more particularly, for enhancing a Th1 response in a mammal and/or suppressing a Th2 response in a mammal. Such animal feeds or animal feed ingredients are also useful for the treatment, prevention, improvement of, and/or alleviation of at least one symptom of a variety of conditions and diseases, including, but not limited to, allergic disease, non-allergic inflammatory disease, viral infection and cancer.

It is still another object of the present invention to provide a topical or cosmetic composition comprising a hardy kiwifruit preparation(s) described herein, formulated together with and/or provided sequentially with one or more steroids, including steroid-based compounds or compositions. Such cosmetic or topical compositions can be used, for regulating an immune response in the mammal, and more particularly for regulating a Th2 and/or a Th1 immune response in a mammal, and even more particularly, for enhancing a Th1 response in a mammal and/or suppressing a Th2 response in a mammal. Such cosmetic compositions are also useful for the treatment, prevention, improvement of, and/or alleviation of at least one symptom of a variety of conditions and diseases, including, but not limited to, allergic disease (including allergic diseases of or affecting the skin), non-allergic inflammatory disease, viral infection and cancer.

Any of the compositions, ingredients, additives, or agents described herein may additionally comprise at least one conventional carrier, adjuvant or diluent. For example, a composition according to the present invention can include pharmaceutically acceptable carriers, adjuvants or diluents. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient(s) after their administration to a patient.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to these carriers. For topical administration, the compounds of the present invention can be formulated in the form of ointments and creams.

Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (CytRx™, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark).

Carriers are typically compounds that increase the half-life of a therapeutic composition in an animal. Suitable carriers include, but are not limited to, polymeric controlled release formulations, biodegradable implants, liposomes, oils, esters, and glycols.

Suitable excipients of the present invention include excipients or formularies that transport or help transport, but do not specifically target an agent or compound to a cell, tissue or site in an animal (also referred to herein as non-targeting carriers). Examples of pharmaceutically acceptable excipients, many of which are also diluents include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions of the present invention can be sterilized by conventional methods and/or lyophilized.

Compositions or formulations of the present invention may be prepared in any suitable form for use in a pharmaceutical or nutraceutical/nutritional/supplement application, such as in an oral dosage form (effervescent tablet, effervescent powder, powder, tablet, capsule, soft capsule, aqueous medicine, syrup, elixirs pill, powder, sachet, granule), or as a topical preparation (cream, ointment, lotion, gel, balm, patch, paste, spray solution, aerosol and the like), or as an injectable preparation (solution, suspension, emulsion). The pharmaceutical and nutraceutical/nutritional/supplement compositions can be formulated for use with any animals, including, but not limited to, humans, companion/domestic animals (e.g., dogs, cats), livestock and other animals.

The composition of the present invention can also be provided, in one embodiment, as a health food (including medical food) that includes the hardy kiwifruit preparations and/or the steroids, including steroid-based compositions of the invention. The health food can be provided as a food product, powder, granule, tablet, chewing tablet, capsule or beverage, etc. Child or infant foods are also included in the compositions of the invention, such as modified milk powder, infant formulas, and modified infant or children's food.

Suitable food products into which a preparation, composition or agent of the invention can be introduced to produce a health food product include, but are not limited to, fine bakery wares, bread and rolls, breakfast cereals, processed and unprocessed cheese, condiments (ketchup, mayonnaise, etc.), dairy products (milk, yogurt), puddings and gelatin desserts, carbonated drinks, teas, powdered beverage mixes, processed fish products, fruit-based drinks (including fruit juices), vegetable-based drinks (including vegetable juices), chewing gum, hard confectionery, frozen dairy products, processed meat products, nut and nut-based spreads, pasta, processed poultry products, gravies and sauces, potato chips and other chips or crisps, chocolate and other confectionery (cookies, candy, licorice), ice creams, dehydrated foods, cut or processed food products (e.g., fruits, vegetables), spices, alcoholic beverages, noodles, fermented foods, soups and soup mixes, soya based products (milks, drinks, creams, whiteners), vegetable oil-based spreads, and vegetable-based drinks. A composition of the present invention can also be used with a food, such as placed onto, poured onto or mixed into the food at the time of serving or when preparing the food.

Compositions, and particularly cosmetic formulations containing the above-identified compositions, may be prepared in any form such as skin, lotion, cream, essence, toner, emulsion, pack, soap, shampoo, rinse, cleanser, body washing solution, washing solution, treatment, gel, balm, spray solution and the like.

It is still another object of the present invention to provide an animal feed or feed ingredient (sometimes also referred to as a feed additive) comprising the hardy kiwifruit preparation(s) described herein and/or the steroid, including steroid-based compositions of the invention. The feed ingredient can be provided as a food product, powder, granule, tablet, liquid, emulsion, paste, etc., which can be formulated into an animal feed product, or applied to or mixed with an animal feed as needed (e.g., a powder to be sprinkled on or mixed into an animal feed, or liquid drops to be added to animal feed). Animal feed products include any feed products or feed additives used for animals, including for domestic pets (companion animals) and livestock, and include, but are not limited to animal food or chow (e.g., dog food, cat food, cattle feed, etc.), biscuits, treats, bones, chews, pastes, tablets, powders, water additives, etc.

Any of the compositions or preparations of the invention can further comprise or be used in conjunction with additional therapeutic or nutraceutical/nutritional agents (additional active ingredients) for the prevention, treatment, improvement, and/or alleviation of at least one symptom of any of the above-described conditions or diseases, including, but not limited to, allergic disease, non-allergic inflammatory disease, viral infection and cancer. Additional active agents other than the hardy kiwifruit preparation(s) and the steroid/steroid-based compound(s) can include pharmacologically active agents and/or nutritionally active agents. Active agents typically contribute at least one additional desirable, nutritional, and/or therapeutic and/or pharmacological property to a composition, in addition to the kiwifruit preparation and/or the steroid composition described herein. Preferred additional active agents include, but are not limited to, anti-inflammatory compounds, anti-allergy compounds, or any other compounds or compositions that can regulate an immune response or provide a benefit to a patient, including by alleviating (reducing, diminishing, decreasing) at least one symptom of a disease or condition such as allergic disease, non-allergic inflammatory disease, viral infection and cancer. Additional agents, including additional active agents, can be included in a composition, preparation, ingredient, additive, or other formulation of the invention in any effective amount. An effective amount is an amount sufficient to achieve the desired effect imparted by the agent, such as an effect on the health or nutrition of a subject (e.g., a therapeutic or nutritional effect), a taste effect, an aroma effect, a visual effect, etc. One of skill in the art will be able to determine the appropriate amount of additional agents to add to a composition of the invention.

For example, compositions of the invention may include one or more therapeutic agents (e.g., medicines), which can also be referred to herein as active agents, used to treat a condition or disease that can be treated or ameliorated by regulation of immune responses, or alleviate a symptom thereof. Such additional therapeutic agents include, but are not limited to, antihistamines (any type, including systemic, topical, inhaled, and including H1 and H2 blockers), antibodies (e.g., anti-IgE, anti-IL-10), antibiotics, cyclosporins, antimycotics, respiratory function controllers, analgesics, β-agonists (long or short acting), leukotriene modifiers (inhibitors or receptor antagonists), cytokine or cytokine receptor antagonists, phosphodiesterase inhibitors, sodium cromoglycate, nedocrimil, theophylline, caffeine, carbobenzyloxy-β-alanyl taurine, inhibitors of T cell function and other anti-inflammatory agents.

Any of the compositions provided by or useful in the present invention can also include one or more natural products as an active agent, including, but not limited to, fatty acids and polyketides; organic acids and miscellaneous small organic compounds; aromatic amino acids and phenylpropanoids; terpenoids and steroids; alkaloids; corrins and porphyrins; linear and cyclic peptides, depsipeptides, and other amino acids derivatives; nucleosides and nucleotides; carbohydrates; proteins, cells and cell fragments; herbal preparations and spices; minerals; sterilizers; seasonings; vitamins; electrolytes; and other natural agents.

Other components (compounds or agents) that may be added to a composition of the invention include synthetic flavoring agents, a coloring agent, a processing agent, an alginic acid or the salt thereof, an organic acid, a protective colloidal adhesive, a pH controlling agent, stabilizer, a preservative, a glycerin, an alcohol, a carbonation agent, or any other essential agent of a formulation (for nutritional or therapeutic use by any method of administration), a food, a feed or feed additive, or a beverage.

Particularly preferred components (active agents) to combine with or add to a composition of the invention include, but are not limited to: probiotics; bacterial cell walls and fragments; whey protein; taurine; alanine; carbobenzyloxy-β-alanyl taurine; fatty acids (e.g., conjugated linolenic acid, eicosapentaenoic acid, docosahexaenoic acid, γ-linolenic acid, α-linolenic acid, dihomo-γ-linolenic acid, stearidonic acid); mono-, di-, and triglycerides (composed of any combination of the fatty acids described above); inositol; turmeric; curcumin; rosemary; quercetin; rosemarinic acid; methylsulfonylmethane (MSM); ginseng; ginger; proanthocyanidin; β-carotene; and any other preparation of a different species of kiwifruit than that used as the primary bioactive component, including any member of Actinidiaceae, and particularly any members of the genus Actinidia, including common kiwi species (e.g., A. chinensis or A. deliciosa) and hardy kiwifruit species (e.g., A. arguta, A. polygama, and A. kolomikta).

Fatty acids and polyketides include, but are not limited to: saturated fatty acids (e.g., α-lipoic acid (R, S, or R,S); unsaturated fatty acids (e.g., conjugated linolenic acid, eicosapentaenoic acid, docosahexaenoic acid, γ-linolenic acid, α-linolenic acid, dihomo-γ-linolenic acid, stearidonic acid); fatty acid esters; monoglycerides, diglycerides, and triglycerides (composed of any combination of the fatty acids described above); acetylenic fatty acids; branched-chain fatty acids; prostaglandins; thromboxanes; leukotrienes; aromatic polyketides; macrolides and polyethers; lipid extracts (e.g., marine oils, Echium oil, borage oil, olive oil); and lecithin.

Organic acids and miscellaneous small organic compounds include, but are not limited to, citric acid; fumaric acid; guaiacol; methylsulfonylmethane (MSM); and ascorbic acid.

Aromatic amino acids and phenylpropanoids include, but are not limited to, aromatic amino acids and benzoic acids (e.g., benzoic acid, gallic acid, gentisic acid, p-hydroxybenzoic acid, protocatechuic acid, vanillic acid, salicylic acid, syringic acid); cinnamic acids (e.g., hydroxytyrosol, curcumin, rosmarinic acid, ar-turmerone, caffeic acid, eugenol, chlorogenic acid, neochlorogenic acid, cinnamic acid, ferulic acid, o-coumaric acid, p-coumaric acid); lignans and lignin; phenylpropenes; coumarins; styrylpyrones; flavonoids (e.g., anthocyanidins, such as delphinidin; proanthocyanidins; catechins such as catechin, epicatechin, and theaflavin; flavonols, such as avicularin, hyperoside, quercitrin, isoquercitrin, kaempferol, myricetin, rutin; flavanones, such as naringenin; chalcones, such as phloretin; isoflavones, such as vitexin); stilbenes; flavonolignans; isoflavonoids; and terpenoid quinines (e.g., K vitamins and tocopherols (vitamin E) such as tocotrienols).

Terpenoids include, but are not limited to, monoterpenes (e.g., β-pinene, borneol, carvacvol, geraniol, thymol, 1,8-cineol, terpineol); iridoids (e.g., monotropein); β-ionone (e.g., thirteen carbon precursor to A vitamins); sesquiterpenes (e.g., caryophyllene, farnesol); diterpenes (e.g., A vitamins); sesterterpenes; triterpenes (e.g., α-amyrin, lupeol, ursolic acid); tetraterpenes; and carotenoids (e.g., lycopene, β-carotene, lutein, astaxanthin, canthaxanthin).

Alkaloids include, but are not limited to, pyrrolidine alkaloids, tropane alkaloids, pyrrolizidine alkaloids, piperidine alkaloids, quinolizidine alkaloids, indolizidine alkaloids, pyridine alkaloids, phenylethylamines, tetrahydroisoquinoline alkaloids, galanthamines, indole alkaloids, β-carboline alkaloids, terpenoid indole alkaloids, quinoline alkaloids, pyrroloindole alkaloids, ergot alkaloids, quinazoline alkaloids, quinoline and acridine alkaloids, imizadole alkaloids, piperidine alkaloids, ephedrines, capsaisins, pyridine monoterpene alkaloids, aconitines, purine alkaloids (e.g., allantoin, caffeine, theophylline).

Corrins and porphyrins include, but are not limited to, B vitamins.

Linear and cyclic peptides, depsipeptides, and other amino acids derivatives include, but are not limited to, simple amino acids and their derivatives (e.g., L-acetyl carnitine, choline, taurine, alanine, carbobenzyloxy-β-alanyl taurine), linear peptides, cyclic peptides (e.g., cyclosporins), cyclic depsipeptides, β-lactams, cyanogenic glycosides, glucosinolates, cysteine sulphoxides.

Carbohydrates include, but are not limited to, monosaccharides (e.g., inositol), polysaccharides (e.g., fructo-oligosaccharides, such as inulin (any chain length); galacto-oligosaccharides; chitin and chitosan).

Other natural materials useful in a composition of the invention include, but are not limited to, proteins (e.g., whey protein and superoxide dismutase); cells and cell fragments (e.g., probiotics, meaning live, intact microorganisms such as, e.g., Lactobacillus spp., bacterial cells and cell wall fragments, fungal/yeast cells and cell wall fragments); herbal preparations and spices (e.g., ginseng, huang, turmeric, rosemary, ginger); minerals (e.g., K, Mg, Ca, Mn, Fe, Cu, Zn, B, Si, Se). Metabolites and derivatives of any of these compounds are also encompassed by the present invention.

Many additional compounds, agents and components may be used in formulating a composition of the invention. Such compounds, agents and components include, but are not limited to, a variety of organic acids, phosphates, antioxidants, water soluble vitamins, lipid soluble vitamins, peptide polymers, polysaccharide polymers, sphingolipids, seaweed extracts, algal extracts, oil ingredients (ester oils, hydrocarbon oils, silicone oils, fluoride oils, animal oils, plant oils), humectants, emollients, surface active agents, organic or inorganic dyes, organic powders, ultraviolet absorbing agent, preservatives, antiseptics, plant extracts, pH controllers, alcohols, pigments, perfumes, refrigerants, antihidrotics, and distilled water. Such ingredients may be added to any of the above-described compositions. In one embodiment, the amount of the other ingredients ranges from 0.01 to 5%, more preferably, 0.01 to 3% in that of total composition. Such ingredients can be obtained by conventional methods disclosed in the literature.

A suitable amount or dose of the hardy kiwifruit preparation of the present invention can include, in one embodiment, an amount of from about 0.1 g to about 10 g per kg body weight of the patient, and preferably, from about 1 to 3 g per kg by weight/day of the hardy kiwifruit preparation. The dose may be administered once per day, several times per day, or in longer increments (e.g., every few days, weekly, monthly, etc.), as desired. In terms of the compositions described herein, the amount of hardy kiwifruit preparation used in the compositions of the present invention should be present between about 0.01% to 100% by weight, and preferably between about 0.01% and about 95% by weight, and more preferably 0.5 to 80% by weight based on the total weight of the composition, including any amount in between 0.01% and 100%, in 0.01% increments. In one embodiment, a pharmaceutical composition of the present invention can contain about 0.01-50% by weight of the hardy kiwifruit preparation based on the total weight of the composition. Suitable amounts or doses can vary depending upon the goal of the administration or the condition or the disease being treated, and also on the weight of the subject, severity, drug form, route and period of administration, and may be chosen by those skilled in the art.

In one embodiment, a hardy kiwifruit preparation used in the present invention may be provided in any composition at from about 20% to 90% highly concentrated liquid, powder, or granule, including any increment between 20% and 90%, in 1% increments.

The ratio of additional components in the composition may generally range from about 0 to 20 w/w % per 100 w/w % of the composition, including any increment between 0 and 100 w/w %, in 1% increments.

In one embodiment, a cosmetic composition comprises the hardy kiwifruit preparation in an amount of from about with 0.01 to 30%, and more preferably, 0.01 to 5% by the weight based on the total weight of the composition, including any increment between 0.01% and 30%, in 0.01% increments.

In another embodiment, a composition comprising the hardy kiwifruit preparation that is added to food, a food ingredient or a beverage, can be provided in an amount ranging from about 0.1 to 95 w/w %, preferably 1 to 80 w/w % of total weight of food, food ingredient or beverage, including any increment between 0.1 and 95 w/w %, in 0.1 w/w % increments, or about 1 to 30 g per 100 ml and preferably 3 to 10 g per 100 ml, including any increment between 1 g per 100 ml and 30 g per 100 ml, in 1 g increments, of a beverage composition.

In one embodiment, a health food of the present invention comprises the hardy kiwifruit preparation of the present invention as 0.01 to 80%, preferably 1 to 50% by weight based on the total weight of the composition, including any increment between 0.01% and 80%, in 0.1% increments.

In one embodiment, a health beverage of the present invention comprises the hardy kiwifruit preparation in an amount of from about 0.01 to about 20% by weight of the total weight of the composition, including any increment between 0.01% and 20%, in 0.01% increments. Providing that the health beverage composition of present invention contains the above-described hardy kiwifruit preparation as an essential component, there is no particular limitation on the other components in the beverage, wherein the other component can be various sweeteners and/or flavor enhancers, such as may be added to a conventional beverage.

A food ingredient can be added to a food by deposition, spray, or mixing. The amount of the added ingredient with respect to the total composition may generally range from about 0.01 to 20 w/w % per 100 w/w % of the present composition, including any increment between 20 w/w % and 100 w/w %, in 1 w/w % increments. Food ingredients or additives can also be mixed with a feed, such as an animal feed, in an amount of from about 5 to 100 g per 1 kg by weight based on the total dried weight of the feed, including any increment between 5 g and 100 g per 1 kg by weight, in 1 g increments.

A suitable dose of a steroid, including steroid-based compound or composition of the invention can include from about 0.001 μg to about 10 mg of steroid per kg body weight of the patient, and can include any dose in between 0.0001 μg and 10 mg per kg body weight, in increments of 0.001 μg (i.e., 0.001 μg, 0.002 μg, 0.003 μg . . . 1.000 μg, 1.101 μg, etc.). Suitable doses of steroids for the treatment of a variety of subjects, including a variety of mammals, are known in the art. Suitable doses are described for humans and dogs in the Examples. It is to be recognized that the present invention allows for a reduction in the amount of steroid to be administered to an individual, and/or a reduction in the duration of treatment and/or frequency of treatment with steroid compositions.

### Methods of the Invention

Accordingly, it is an object of the present invention to provide a method to selectively regulate immune responses in a patient (individual), and more particularly, for regulating a Th2 and/or a Th1 immune response in a mammal, and even more particularly, for enhancing a Th1 response in a mammal and/or suppressing a Th2 response in a mammal. Such methods are useful for the treatment of, prevention of, improvement of, and/or alleviation of at least one symptom of, a variety of conditions and diseases, including, but not limited to, allergic disease, non-allergic inflammatory disease, viral infection and cancer. Such methods include administration of or provision of a composition or compositions of the invention as described herein, to an individual (patient, subject, animal) that would benefit from such administration or provision of the composition or compositions of the invention. In particular, the method of the invention includes the administration of or provision of a composition that comprises, consists essentially of, or consists of, a kiwifruit preparation and one or more steroids, including steroid-based compounds or compositions. Alternatively, a kiwifruit preparation is administered or provided together with and/or sequentially with (before or after) a second composition comprising, consisting essentially of, or consisting of, one or more steroids, including steroid-based compounds or compositions. Sequential administrations can be contemporaneous (within the same administration dosage period) or separated by days, weeks, or months, as needed in order to achieve the desired result. Preferably, the steroid and hardy kiwifruit composition are used together in the same composition or are used contemporaneously. In one embodiment, periods of combined use (hardy kiwifruit and steroid) can be followed or preceded by periods of monotherapy with the kiwifruit preparation alone. The compositions of the invention are described in detail above.

In particular embodiments of the invention, the administration of the combination of kiwifruit preparation and steroid preparation alleviates (reduces, diminishes, provides relief from, decreases, etc.) at least one, and preferably more than one, symptom of a disease or condition associated with inflammation and particularly, allergic inflammation. Such symptoms include, but are not limited to, itching (pruritus), redness (erythema), oozing and crusting (excoriations), thickening (lichenification), hair loss (alopecia), and/or swelling of the skin in a mammal. Any other symptoms of inflammation can also be alleviated using the present invention. The present inventors have discovered that the combined use of a hardy kiwifruit preparation of the invention with conventional steroid therapy has a synergistic effect on reducing symptoms of allergic disease, such as allergic dermatitis and at a minimum, that the use of a hardy kiwifruit preparation of the invention serves as an effective adjunctive therapy to conventional steroid therapy in the treatment of allergic disease, such as allergic dermatitis.

The administration or provision of the composition, and particularly the combination therapy described herein (hardy kiwifruit preparation and steroid-based therapy), may also result in at least one of the following detectable or measurable results, expressed here as biological activities: (a) reduces the number of IgE-producing B cells in a patient; (b) reduces the amount of IgE produced in a patient (e.g., in the serum or plasma); (c) decreases production and/or levels of at least one Th2 cytokine (e.g., IL-4, IL-5, IL-10); (d) increases the level of at least one Th1 cytokine (e.g., IL-12, IFN-γ); (e) decreases the level of expression of the transcription factor, GATA-3; (f) increases the level of expression of the transcription factor T-bet; (g) increases the level of expression of the transcription factor NFATc2; (h) increases the number of IgG2a-producing B cells in a patient; (i) increases the amount of IgG2a produced in a patient; (j) enhanced production or activity of Th1 T lymphocytes (e.g., CD4+, IFN-γ+), particularly at a site of inflammation; (k) decreased production or activity of Th2 T lymphocytes (e.g., CD4+, IL-4+), particularly at a site of inflammation; (l) reduces the number of IgG1-producing B cells in a patient; (m) reduces the amount of IgG1 produced in a patient; and/or (n) reduces the level of or production of at least one leukotriene in the patient. An improvement in the health of the individual or benefit to the individual as a result of administration or provision of the composition(s) of the invention, particularly as measured by an alleviation of any symptom of allergy or inflammation, any beneficial change in the immune response of the individual, and/or detection of any other beneficial biological activity described herein, is encompassed by the invention and is a useful result of the compositions and methods of the invention.

In one aspect of the invention, the administration or provision of a composition(s) of the invention, and particularly the combination therapy described herein (hardy kiwifruit preparation and steroid-based therapy), reduces leukotriene production in a patient, thereby treating or ameliorating at least one symptom of a condition or disease associated with leukotrienes in a patient. Diseases and conditions associated with leukotrienes include, but are not limited to, asthma, food allergy, allergic rhinitis, chronic urticaria, and allergic dermatitis. In this embodiment, preferred routes of administration include oral, inhaled and topical administration, in addition to systemic routes of administration.

Accordingly, the method described above can be used for the prevention and/or treatment of any disease or condition, and/or the alleviation of any symptom thereof, in which regulation of the immune response in the manner described herein would be, or could be predicted to be, beneficial to a patient. The invention also includes the administration or provision of compositions of the invention to reduce the amount and frequency of the use of steroid therapy and/or any other therapy, particularly conventional therapies, in a patient.

It is therefore an object of the present invention to provide a method of treating and/or preventing allergic disease and non-allergic inflammatory disease in a mammal, comprising administering to said mammal an effective amount of any of the hardy kiwifruit preparations described herein, together with (concurrently and/or sequentially) one or more steroids, including steroid-based compounds or compositions. Such compositions typically include a pharmaceutically acceptable carrier

As used herein, the phrase “protected from a disease” refers to reducing (alleviating) the symptoms of the disease; reducing the occurrence of the disease, and/or reducing the severity of the disease. Protecting a patient can refer to the ability of a composition(s) of the present invention, when administered to a patient, to prevent a disease from occurring and/or to alleviate at least one, and preferably more than one, disease symptoms, signs or causes. Preferably, as a result of alleviation of symptoms, the patient has an improved quality of life, has improved health, can reduce the use (amount and/or frequency) of potentially harmful, unpleasant or otherwise deleterious therapies, and/or no longer experiences the disease or condition to the extent prior to the use of the compositions and methods of the invention, which can include complete treatment of the disease. As such, to protect a patient from a disease includes both preventing disease occurrence (prophylactic treatment) and treating a patient that has a disease (therapeutic treatment), most typically to reduce the symptoms of the disease, rather than cure the disease. In particular, protecting a patient from a disease or enhancing another therapy is accomplished by regulating a given activity such that a beneficial effect is obtained. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

According to the present invention, allergic diseases can include, but are not limited to, asthma, allergic bronchopulmonary aspergillosis, allergic bronchitis bronchiectasis, hypersensitivity pneumonitis, allergic sinusitis, anaphylaxis, allergic rhinitis, allergic conjunctivitis, allergic dermatitis, atopic dermatitis, contagious dermatitis, chronic urticaria, insect allergies, food allergies and drug allergies.

In one embodiment, the allergic disease is atopic dermatitis. In this embodiment, in addition to administration of the composition comprising, consisting essentially of or consisting of a hardy kiwifruit preparation of the invention, the patient is concomitantly treated (by concurrent or sequential administration) with a conventional steroid therapy for atopic dermatitis. In this embodiment of the invention, the hardy kiwifruit preparation and/or the steroid composition is administered most preferably by oral or topical administration, although the invention is not limited to such routes of administration.

In another embodiment, the allergic disease is asthma. In this embodiment, in addition to administration of the composition comprising, consisting essentially of or consisting of a hardy kiwifruit preparation of the invention, the patient is concomitantly treated (by concurrent or sequential administration) with a conventional steroid therapy for asthma, including but not limited to, an inhaled steroid medication. Other asthma controllers may also be used. In this embodiment of the invention, the hardy kiwifruit preparation is administered most preferably by oral or inhaled administration, although the invention is not limited to such routes of administration. Steroid therapy is administered by any suitable method, and most preferably by oral or inhaled administration.

According to the present invention, non-allergic skin inflammation diseases can include, but are not limited to, various skin troubles caused by inflammation such as pimples, acne and the like. The above-described compositions, including when administered concomitantly with a conventional steroid-based therapy, are useful for preventing, treating, and/or improving skin inflammation in a patient.

Other non-allergic inflammatory diseases that can be prevented or treated using the compositions and methods described herein include, but are not limited to, various dermatitis conditions, systemic lupus erythematosus (SLE), retinal inflammation, gastritis, retinopathy, hepatitis, enteritis, pancreatitis, nephritis and similar conditions where reduction of a Th2 type immune response and/or enhancement of a Th1 type immune response would be beneficial. As above, the above-described compositions, particularly when administered concomitantly with or sequentially with a conventional steroid-based therapy, are useful for preventing, treating, and/or improving such diseases, conditions, or symptoms thereof.

In addition to administration of a steroid, including steroid-based compound or composition, in any of the above methods to treat or prevent a disease or condition, the hardy kiwifruit preparation can be administered in conjunction with (concurrently and/or sequentially) any other therapy or composition that is useful for treating the particular condition. In these embodiments, the combination of the hardy kiwifruit preparation and steroid therapy with the additional compositions or therapies may serve as an adjunct to a conventional therapy, to enhance the improvement, recovery, or amelioration of symptoms in the patient. However, it is also recognized by the present inventors that the combination of the hardy kiwifruit preparation and steroid therapy with the additional compositions or therapies can also provide a synergistic effect over the use of any of the therapeutic approaches alone, as has been discovered for the combination of hardy kiwifruit preparation and steroid therapy. In addition, the combination of the use of hardy kiwifruit preparation(s) as described herein with steroid therapies, provides the advantage of being able to decrease the amount per dose and/or frequency of administration of the steroids, and may additionally allow decreased amount and frequency of administration of other therapies used concurrently with the hardy kiwifruit/steroid therapy of the invention. Since steroids and other therapies are often associated with undesirable side effects, the ability to reduce the use of such components provides great benefits to a patient.

Particularly preferred types of additional conventional agents or therapies that can be used together with a hardy kiwifruit preparation of the invention include, but are not limited to, fatty acids (particularly longer chain fatty acids, including fatty acids with 18 and higher carbon chains), antihistamines (any type, including systemic, topical, inhaled), antibodies (e.g., anti-IgE, anti-IL-10), antibiotics, cyclosporins, antimycotics, respiratory function controllers, analgesics, β-agonists (long or short acting), leukotriene modifiers (inhibitors or receptor antagonists), cytokine or cytokine receptor antagonists, phosphodiesterase inhibitors, sodium cromoglycate, nedocrimil, caffeine, theophylline, carbobenzyloxy-β-alanyl taurine, ginger, curcumin, catechins, flavanols, flavonoids, polyphenols, inhibitors of T cell function and other anti-inflammatory agents.

In the method of the present invention, compositions can be administered or provided to any member of the Vertebrate class, Mammalia, including, without limitation, primates, rodents, livestock, horses and domestic pets. Preferred patients to protect are domestic pets (e.g., companion animals, including, but not limited to, dogs and cats) and humans, with humans being particularly preferred. All modes of administration are contemplated. According to the present invention, the terms “patient”, “subject” and “individual” can be used interchangeably.

According to the present invention, an effective administration protocol (i.e., administering a composition in an effective manner) comprises suitable dose parameters and modes of administration that provide the desired result with respect to the regulation of inflammation or a symptom of a disease or condition in the patient. Effective dose parameters can be determined using methods standard in the art for a particular disease or condition. Such methods include, for example, determination of survival rates, side effects (i.e., toxicity) and progression or regression of disease.

Compositions of the invention can be administered 2, 3, 4 or more times daily; once per day (daily); 2, 3, 4, 5, or 6 times per week; weekly; bi-weekly; monthly; or at any other interval that is deemed to be effective. Administration can be on an as-needed basis, for a defined period of time (typically measured in several days, weeks or months), or chronically for the life of the patient.

In one aspect of the invention, the method includes a period of administration of a combination of the hardy kiwifruit preparation contemporaneously with a steroid, including steroid-based compound or composition, either in the same (administered concurrently) or separate compositions (administered sequentially), which is preceded by and/or followed by a period of administration of the hardy kiwifruit preparation alone. During the period of contemporaneous administration, the hardy kiwifruit preparation can be administered at substantially the same time as the steroid, including steroid-based compound or composition (e.g., at the same or approximately the same time(s) each day), or at different times within the same administration schedule (e.g., at different times within the same day, if the single administration schedule is daily). The period of administration of combination therapy versus monotherapy can be determined based on responsiveness of the patient, but may include alternating intervals of 1 week (e.g., daily administration of combination therapy for one week, followed by daily administration of monotherapy for 1 week, repeated as needed), 2 weeks, 3 weeks, 4 weeks, 5 weeks, 6 weeks, 7 weeks, 8 weeks, 1 month, 2 months, 3 months, 4 months, 5 months, 6 months, and up, as determined by the clinician. Alternating administration periods need not be the same length of time. For example, combination therapy can be administered for one period of time (e.g., 4 weeks), and then monotherapy can be administered until continued improvement or maintenance of improvement of symptoms is no longer observed, which may be several weeks or months. Subsequently, combination therapy can be administered as needed. Moreover, dosage requirements for the kiwifruit preparation may differ from the dosage requirements of the steroid-based therapy, but this is within the ability of the clinician to determine.

Administration routes include in vivo and ex vivo routes, but most commonly includes in vivo administration. Ex vivo refers to performing part of the regulatory step outside of the patient. In vivo routes include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, aural, intranasal, oral, pulmonary administration, impregnation of a catheter, intracutaneous, intrathecal, epidural, intracerebroventricular injection, and direct injection into a tissue. In one embodiment of the present invention, a composition is administered by a parenteral route (e.g., subcutaneous, intradermal, intravenous, intramuscular and intraperitoneal routes). Intravenous, intraperitoneal, intradermal, subcutaneous and intramuscular administrations can be performed using methods standard in the art. Aural delivery can include ear drops, intranasal delivery can include nose drops or intranasal injection, and intraocular delivery can include eye drops. Aerosol (inhalation) delivery can also be performed using methods standard in the art (see, for example, Stribling et al., Proc. Natl. Acad. Sci. USA 189:11277-11281, 1992, which is incorporated herein by reference in its entirety). For example, in one embodiment, a composition or vaccine of the invention can be formulated into a composition suitable for nebulized delivery using a suitable inhalation device or nebulizer. Oral delivery can be performed by complexing a composition of the present invention to a carrier capable of withstanding degradation by digestive enzymes in the gut of an animal, for example, as tablets or capsules, as well as being formulated into food, feed and beverage products. Examples of such carriers, include plastic capsules or tablets or food stuffs, such as those known in the art. Direct injection techniques are particularly useful for site-specific administration of a compound. Oral delivery or topical delivery are particularly preferred routes of delivery or administration according to the present invention. Routes of administration that modulate mucosal immunity are useful in the treatment of viral infections and some allergic conditions. Such routes include bronchial, intradermal, intramuscular, intranasal, other inhalatory, rectal, subcutaneous, topical, transdermal, vaginal and urethral routes.

The present invention is more specifically explained by the following examples. However, it should be understood that the present invention is not limited to these examples in any manner.

### EXAMPLES

### Example 1

The following example shows the preparation of various preparations comprising A. arguta that were used in the examples below.

### Plant Material

Stems (consisting of canes and fruiting spurs), roots, and bark of Actinidia arguta (Sieb. Et Zucc.) Planch. ex Miq. (Actinidaceae) cultivar ‘Ananasnaya’ were collected at Hurst Berry Farm, Sheridan, Oreg. A voucher specimen (#518640) was authenticated by Mr. Tim Hogan, Collection Manager, University of Colorado Herbarium, The University of Colorado, Boulder, Colo., and deposited at the same location. Plant material was air dried 48 hours and stored at room temperature prior to extraction or other processing.

Ripe, ready-to-eat A. arguta fruit were collected at Hurst Berry Farm, frozen immediately, shipped and stored frozen (−20° C.) prior to extraction or other processing.

### Extracts and Other Preparations

Powdered stems (126.6 g), powdered roots (79.0 g), and finely divided bark (126.2 g) were each extracted with distilled water (1 L) at 94° C. for 4 h. The mixtures were then filtered, and the filtrate concentrated to dryness by rotary evaporation to provide a stem extract (9.9 g), a root extract (8.6 g), and a bark extract (2.4 g).

Twenty frozen A. arguta berries (154.4 g) were thawed at room temperature, crushed, and extracted with distilled water (1 L) 91° C. for 5 h. The mixture was filtered, and the filtrate concentrated to produce a ‘boiled’ fresh fruit extract (12.8 g).

Additional fresh-frozen kiwifruit (341.6 g) was thawed and run through a juicer. The juicer removed the skins from the fruit resulting in a mixture of seeds, pulp, and juice. This mixture was centrifuged (30 min., ˜3500 rpm) to provide 150 mL of juice. This juice was concentrated to dryness by rotary evaporation resulting in a fruit juice concentrate (24.2 g).

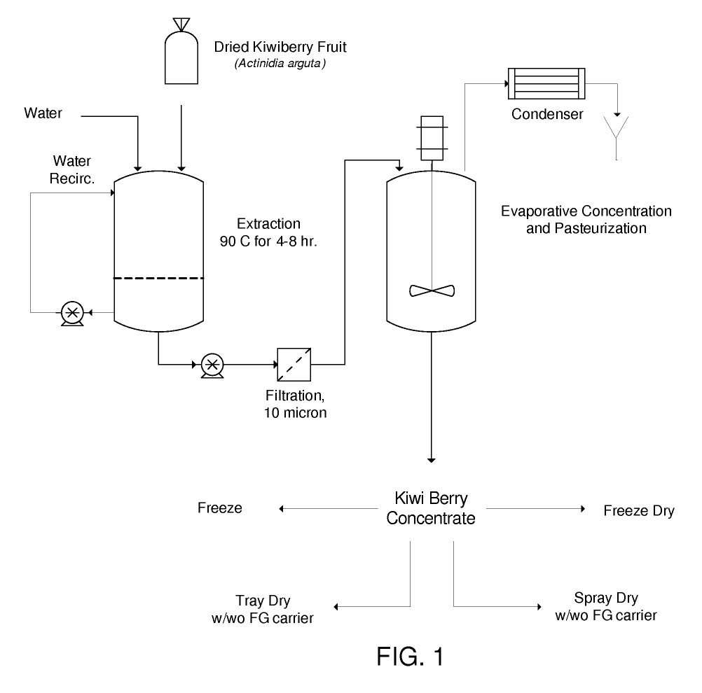

In order to generate larger quantities of an extract equivalent to that described in U.S. Patent Publication No. 2004/0037909, supra, a process scale extraction of the kiwifruit was performed (Sungil Bioex Co., Ltd., Bibong, Korea). Frozen kiwifruit (1242 kg) were sliced (¼″ to ⅜″ thickness) and dried in a convection dryer (65-80° C.) to a moisture content of 5-20%. Batch extraction (FIG. 1) of the dried fruit (239 kg) was performed in a jacketed stainless steel reactor with an internal filter screen to support the extraction load. An external condenser was employed to prevent water loss during the extraction. The quantity of extraction solvent (water) was based on 5-10 times the weight of the dried fruit to be extracted. The contents of the extraction vessel were heated from 0 to 90° C. over a period of 2 h via the introduction of steam into the jacket of the reactor. Water (90° C.) was then recirculated through the biomass using an external recirculation loop for 4-12 h. Subsequently, the spent biomass was removed for disposal and the aqueous extract filtered through a 10 micron filter. The filtrate was then concentrated under vacuum (˜600 mmHg) at 55-65° C. in an agitated stainless steel reactor equipped with an external condenser and a distillate receiver. Once the material was concentrated, it was held at 80° C. for an additional 30 min. to sterilize the extract. The resulting material (101 kg), equivalent to PG102T, was designated FD001. Of this material, 3 kg were set aside for further testing. Good Manufacturing Practices were used throughout the process.

To create a powdered material appropriate for encapsulation and useful in the clinical applications described herein, the FD001 concentrate produced above (98 kg) was pumped to a horizontal paddle blender and mixed with an equal weight, based on the calculated solids content, of microcrystalline cellulose (MCC). Following this, the solid blend was transferred to stainless steel trays that were placed into a forced hot air dryer (70-80° C.) for 24 h. The dry, lumpy solids were then ground in a Fitzmill type hammer mill to produce a 40 mesh powder (118 kg). This material was encapsulated (GMP Laboratories of America, Inc., Anaheim, Calif.) into 300 mg- or 600 mg-sized capsules, each containing a 1:1 mixture of FD001 and MCC for use in canine and human clinical trials.

Dried A. arguta fruit (7.0 g) from process-scale material, sliced and dried as in the initial steps above (but not subjected to the batch extraction), was powdered and this material was extracted with water (250 mL) 25° C. for 4 hours. The mixture was filtered and the filtrate concentrated to dryness by rotary evaporation to provide a room temperature water extract of the dried fruit (4.2 g).

FD001 (79.9 g) was blended with 1.5 L distilled H₂O and this solution extracted successively with four 500 mL portions of ethyl acetate (EtOAc). The combined organic layers and the aqueous layer were concentrated to dryness in vacuo resulting in an EtOAc extract (7.4 g) and the aqueous remainder (41.5 g).

FD001 (21.45 g) was dissolved in DI water and filtered. The Filtrate was loaded on to an equilibrated C-18 reverse phase packing in a gravity column approximately 4.5 cm (i.d.) by 10 cm. The column was eluted with 500 ml water (Cut F1) followed by 500 ml 25% methanol in water (Cut F2), 500 ml of 50% methanol in water (Cut F3), 5000 ml of 75% methanol in water (Cut F4), 500 ml of 100% methanol (Cut F5) and 500 ml of 100% DCM (Cut F6). The eluents were collected and evaporated to dryness in vacuo, yielding 14.16 gm Cut F1, 690 mg Cut F2, 230 mg Cut F3, 30 mg Cut F4, 83 mg Cut F5 and 350 mg Cut F6. The overall recovery was 72.4% by weight.

### Example 2

The following example describes in vitro testing for immunomodulating activity in A. arguta preparations.

The purpose of this study was to compare the relative ability of various extracts and preparations produced from A. arguta to modulate cytokine production (IL-4, IL-5, IL-10, IL-13, and IFNγ) in splenocyte cultures derived from ovalbumin (OVA, grade V, Sigma)-sensitized mice using ELISA (Quantikine kits, R&D systems) analysis. The following samples (prepared as described in Example 1 above) were tested: FD001 (PG102T), the fruit juice concentrate, and the EtOAc extract.

### Splenocyte Isolation and Culturing

Female, Balb/c mice (Harlan, Indianapolis, Ind.) were sensitized by IP injection of 20 μg OVA on days 0 and 14. On day 24, following euthanasia by cervical dislocation, spleens were aseptically removed from individual mice and immediately processed for splenocyte culture development using sterile technique. The spleens were dissociated in the presence of 10 mM HEPES-buffered RPMI-1640, by gently forcing the tissues through the grid of a 70 micron nylon mesh using the plunger from a 3 cc syringe. Large cell aggregates were removed from the resulting suspension using a FCS-gradient. The splenocytes were then centrifuged (1500 rpm, 5 min.) and the resulting cell pellets were treated with RBC lysis buffer (10 min., RT) to remove the contaminating erythrocytes. The majority of the RBC lysis buffer was then removed by centrifugation (1500 rpm, 5 min.) and the pelleted splenocytes were then washed 3× with 10 mM HEPES-buffered RPMI-1640. Following the final wash, the pelleted splenocytes were resuspended in a volume of in RPMI-1640 containing 10% FCS and Penn/Strep (complete medium) designed to deliver a final cell density of 5×10⁶ cells/mL. For each analysis, 5×10⁶ splenocytes were plated into the individual wells of a 24-well plate. On day 3, the supernatants from these wells were collected and frozen in preparation for the determination of experimental results.

Control splenocyte cultures were also established from naïve (non-sensitized) mice in the manner described above and plated out into the individual wells of a 24-well plate to achieve a final cell density of 5×10⁶ cells/mL. These splenocytes were established in RPMI-1640 containing 10% Fetal Calf Serum, Penn/Strep, and they received no additional treatment. On day 3, the supernatants from these wells were collected and frozen to serve as negative experimental controls.

Stimulation of Splenocyte Cultures with A. arguta Preparations

Ten OVA-sensitized mice were used for each preparation tested. Splenocytes from each mouse were plated (5×10⁶ cells/mL) into 8 individual wells of a 24-well plate in complete RPMI-1640 medium containing 100 μg/mL OVA, 0.5% DMSO and either no or chosen concentrations of each of the specific test preparations under examination. 6 of the 8 wells were partitioned into 2 sets of 3 wells. Each of the wells in the sets of 3 were treated with A. arguta preparations at concentrations of either 0.25, 1.0 or 10 mg/mL. To serve as positive controls, the 7^{th} wells were treated with 2 μM dexamethasone (DEX), a potent glucocorticoid anti-inflammatory. The 8^{th} wells received no additional treatment and served as an OVA-only experimental control. After 3 days of culture, the supernatants from each of the unique 8 wells per OVA-sensitized mouse were collected and frozen. These supernatants were used to determine the levels of the cytokines IL-4, IL-5, IL-10, IL-13, and IFN-γ present in the culture medium.

### Determination of Cytokine Levels in Culture Supernatants

The cytokine levels in the culture supernatants derived from the A. arguta preparation-treated splenocytes, DEX-treated splenocytes, OVA-only treated splenocytes, and untreated splenocytes from non-sensitized control mice were determined by ELISA assay. Two replicate ELISA plate wells were utilized for each cytokine level determination.

### Results

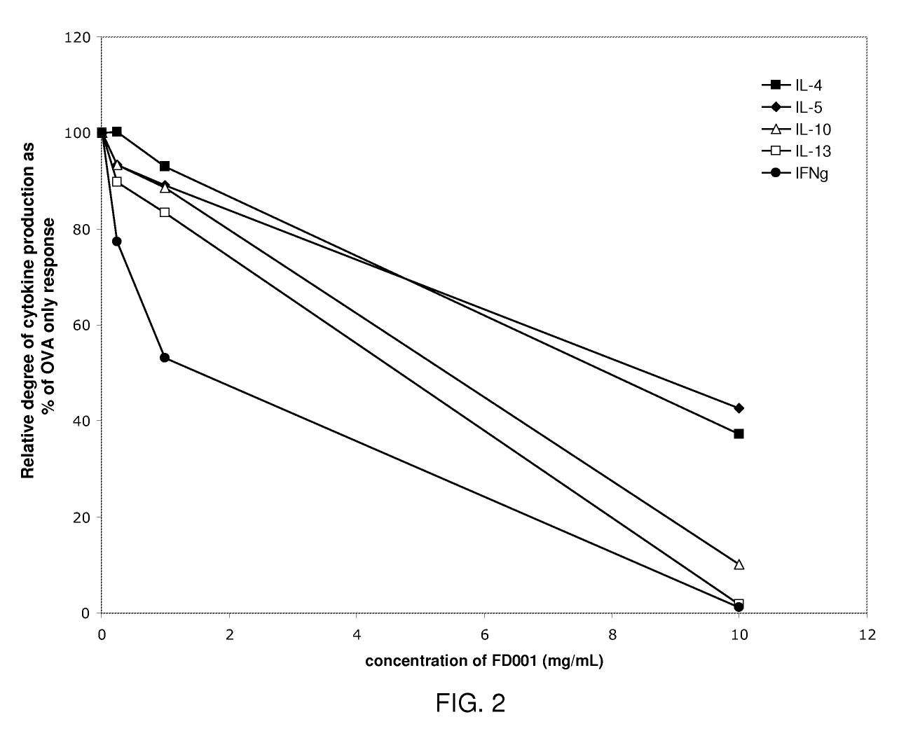

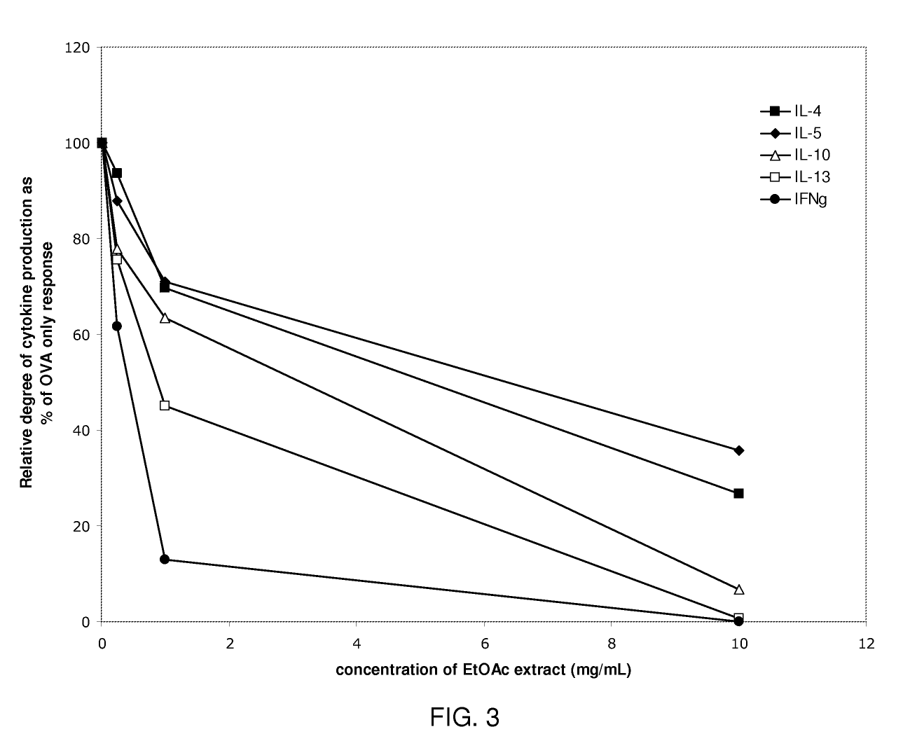

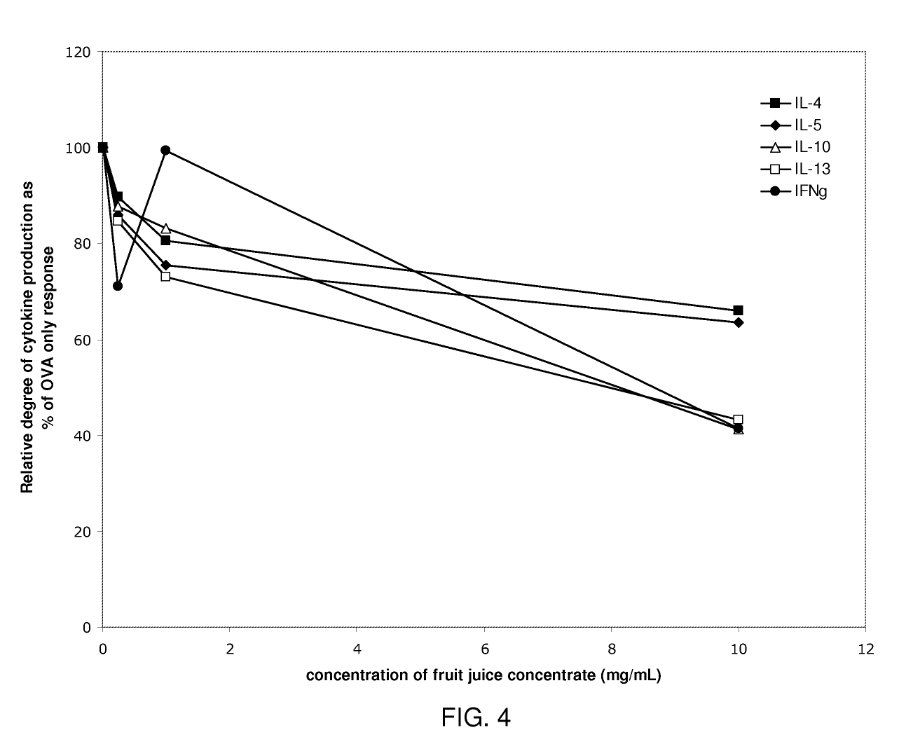

This in vitro work confirmed that the activity in FD001 (FIG. 2), the EtOAc extract (FIG. 3), and the fruit juice concentrate (FIG. 4) of A. arguta were substantially similar to that described for the hardy kiwifruit fruit extract described in U.S. Patent Publication No. 2004/0037909, supra. It was observed that all three preparations (at 10 mg/mL) caused substantial suppression, to varying degrees, of the cytokines IL-4, IL-5, IL-10, IL-13, and IFNγ. The most pronounced effects were seen on IL-13 and IFN-γ for all of the samples examined, consistent with prior in vitro work described in U.S. Patent Publication No. 2004/0037909, supra. Since activity was observed in the EtOAc extract, it is evident that active constituents present in FD001 are extractable into organic solvents, and may be further purified by traditional chromatographic methods. Significantly, the fruit juice concentrate also suppressed cytokine production by the splenocytes, indicating that extraction of the kiwifruit as shown in U.S. Patent Publication No. 2004/0037909, supra is not the sole requirement to produce active preparations of hardy kiwifruit. It is noted that relatively less suppression of cytokines was apparent in the fruit juice concentrate, suggesting that the drying or heating process used to prepare FD001 may be important for enhancement of activity.

### Example 3

The following example describes a comparison of in vitro activity of extracts of non-fruit parts of A. arguta, as well as alternative fruit preparations of A. arguta.

The purpose of this study was to assess the ability of A. arguta extracts that originate from plant parts other than the fruit, or from alternative preparations of the fruit (i.e., other than the extracts described in U.S. Patent Publication No. 2004/0037909, supra), to modulate cytokine production (IL-13 and IFNγ) in splenocyte cultures derived from ovalbumin-sensitized mice, using ELISA analysis. The following samples (prepared as described above) were tested: water extracts of the stem, root, and bark of A. arguta, prepared as described in Example 1; “boiled” fresh fruit preparations; the fruit juice concentrate prepared as described in Example 1; FD001 (large scale equivalent of PG102T) prepared as described in Example 1; FD001 powder prepared as described in Example 1 (used for clinical trials described below); a room temperature water extract of the dried A. arguta fruit; the EtOAc extract prepared as described in Example 1; and aqueous remainder, also described in Example 1. In addition, the activity of three known immunosuppressive compounds, cyclosporin, dexamethasone, and quercetin were evaluated as controls.

### Splenocyte Isolation and Culturing

Preparation of the splenocytes was performed in a manner identical to that described above for Example 2.

Stimulation of Splenocyte Cultures with A. arguta Extracts

Splenocyte cells from 8 OVA-sensitized mice (8 replicates) were utilized for the analysis of each extract or preparation tested. 5×10⁶ cells splenocyte cells from each mouse were plated out into the individual wells of a 24-well plate in complete RPMI-1640 medium containing 100 μg/mL OVA and 25 mM HEPES (pH 7.3), 1 ml per well. Kiwifruit preparations were examined at concentrations of 1.0, 3.0, and 10 mg/mL.

### Cyclosporin, Quercetin, and Dexamethasone Analysis

Splenocytes from 8 OVA-sensitized mice (8 replicates) were utilized for the analysis of each compound tested, with the exception of Quercetin where the splenocytes derived from only 2 OVA-sensitized mice were examined. 5×10⁶ cells splenocytes from each mouse were plated out into the individual wells of a 24-well plate in complete RPMI-1640 medium containing 100 μg/mL OVA and 25 mM HEPES (pH 7.3), 1 ml per well. Cyclosporin was examined at concentrations of 0.0083, 0.083, and 4.15 μM. Dexamethasone was examined at concentrations of 0.01, 0.1 and 1 μM. Quercetin was examined at concentrations of 1, 10, and 25 μM. Wells treated with 1 μM dexamethasone, a potent glucocorticoid anti-inflammatory, served as positive experimental controls. Wells receiving only complete RPMI-1640 containing 100 μg/ml OVA and 25 mM HEPES (pH 7.3) served as OVA-only experimental controls. After 3 days of culture, the supernatants were collected and frozen. These supernatants were used to determine the levels of IL-13, and IFN-γ present in the various culture media, under the experimental conditions examined.

### Determination of Cytokine Levels in Culture Supernatants

The cytokine levels in the culture supernatants from all treatment and control wells were determined by ELISA assay. Two replicate ELISA plate wells were utilized for each cytokine level determination.

Based on the results of the in vitro testing described in Example 2, only the expression of IL-13 and IFN-γ were analyzed for the purpose of estimating levels of activity present in the materials tested.

### Results

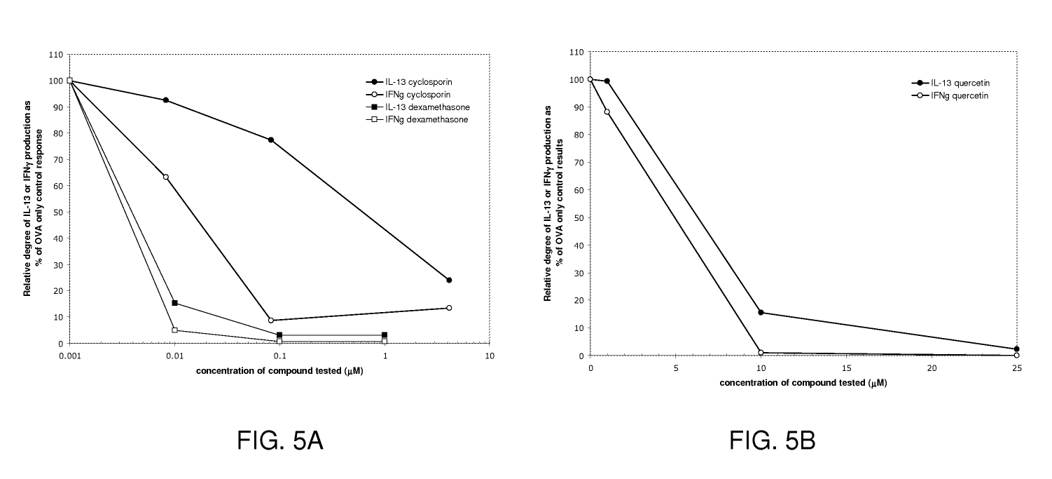

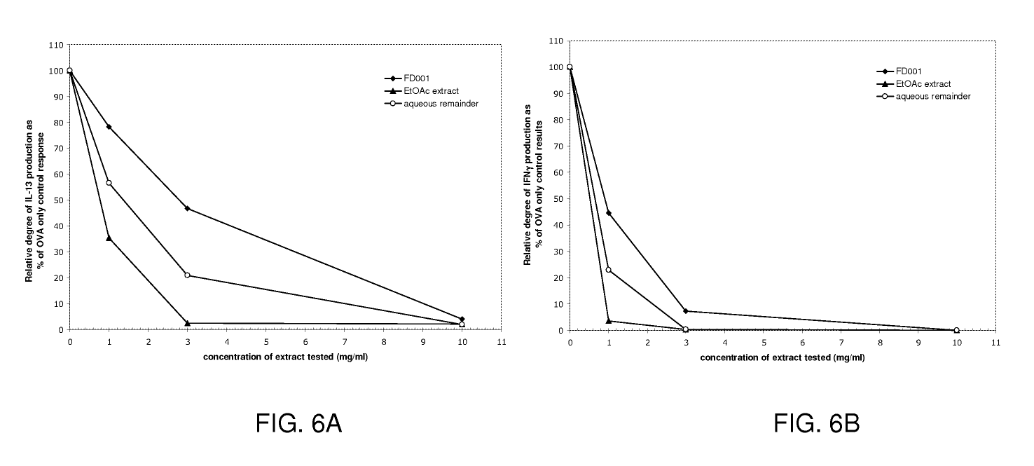

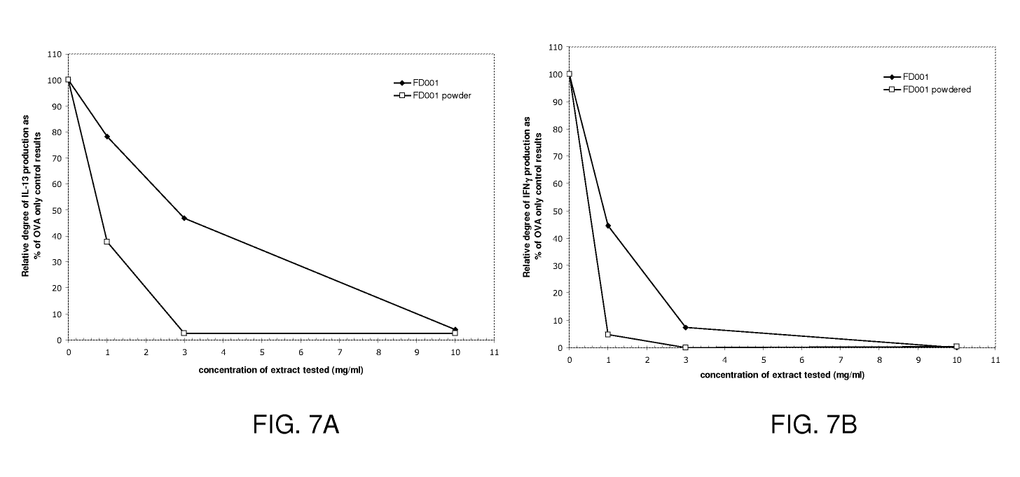

As demonstrated earlier, a greater suppression of the cytokines examined was observed as the concentrations of the A. arguta test materials were increased. In general, suppression was more pronounced against IFNγ. The activity of prescribed immunosuppressant compounds was similar to the A. arguta preparations in this assay. The peptide cyclosporin and the glucocorticoid steroid dexamethasone exhibited potent activity (<1 μM) as shown in FIG. 5A. The flavonoid quercetin showed potent activity over a slightly higher concentration range (1-25 μM, FIG. 5B). Further confirmation of the ability of EtOAc to extract activity from FD001 is demonstrated in FIGS. 6A and 6B. Interestingly, activity was also observed to be present in the aqueous remainder, indicating that both polar and non-polar components may be responsible for the in vitro immunosuppressive effect. The FD001 powder, which was the material used in both canine and human clinical trials (described below), was confirmed to be active in this assay as shown in FIGS. 7A and 7B.

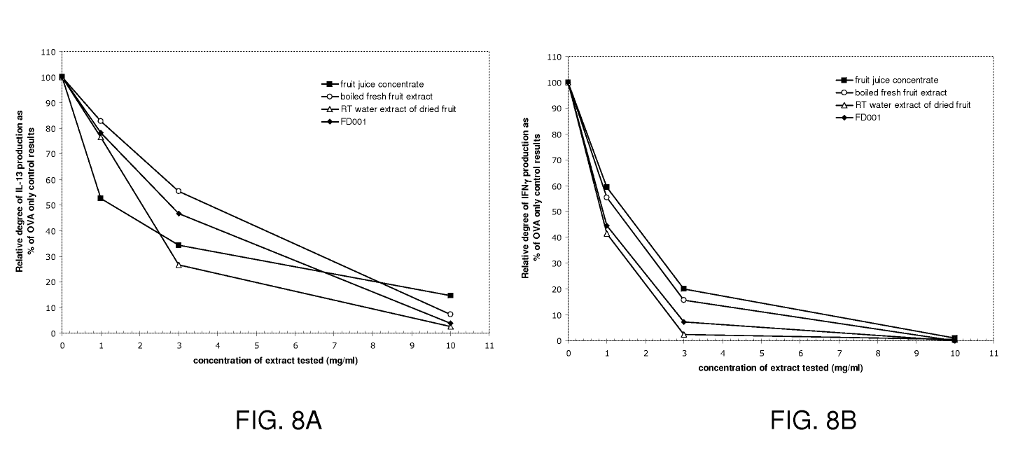

As described in Example 2, alternative methods of preparing the kiwifruit extracts, other than the procedures described in U.S. Patent Publication No. 2004/0037909, supra, were explored. All of the fruit-derived extracts, whether dried or fresh, or extracted in hot or room temperature water, exhibited similar activity in this assay as seen in FIGS. 8A and 8B. Based on this analysis, the present inventors believe that there are several viable alternative methods for preparing hardy kiwifruit for therapeutic purposes.

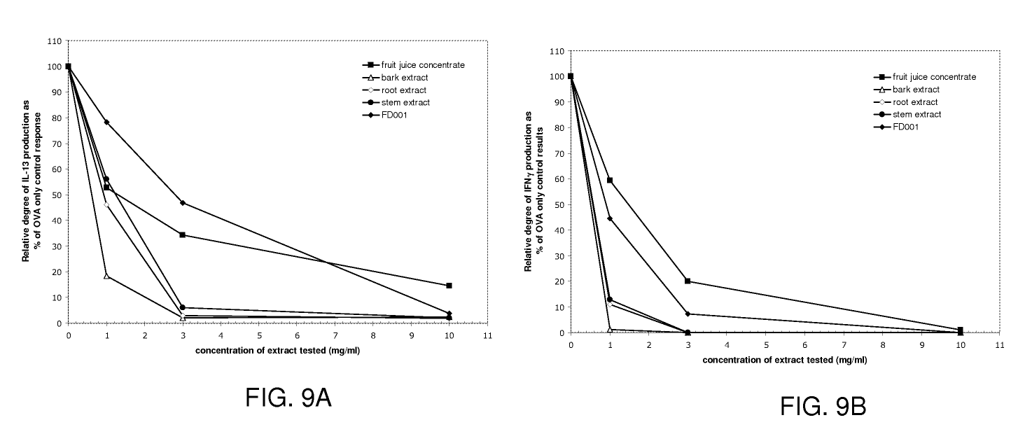

Interestingly, the hot water extracts prepared from the bark, root, and stem of A. arguta exhibited equal or superior activity in this assay (FIGS. 9A and 9B) when compared to FD001 and the concentrate of the fruit juice. These results indicate that these other plant parts may represent alternative sources of compounds of therapeutic interest with regard to modulation of immune markers or suppression of pro-inflammatory cytokines.

### Example 4

The following example describes a comparison of in vitro activity of fractions collected from the C-18 chromatographic purification of FD001.

The purpose of this study was to assess the biological activity of fractions of FD001 after purification through a C-18 chromatographic column eluting with decreasing solvent polarity to modulate cytokine production (IL-13 and IFNγ) in splenocyte cultures derived from ovalbumin-sensitized mice, using ELISA analysis. The following samples (prepared as described above) were tested: fraction cuts F1, F2, F3, F4, F5 and F6. In addition, the activity of three known immunosuppressive compounds, cyclosporin, dexamethasone, and quercetin were evaluated as controls along with a reference control sample of SG05-0217A, equivalent to FD001.

### Splenocyte Isolation and Culturing

Preparation of the splenocytes was performed in a manner identical to that described above for Example 3.

Stimulation of Splenocyte Cultures with A. arguta Extracts

Splenocyte cells from OVA-sensitized mice (8 replicates) were utilized for the analysis of each extract or preparation tested. 5×10⁶ cells splenocyte cells from each mouse were plated out into the individual wells of a 24-well plate in complete RPMI-1640 medium containing 100 μg/mL OVA and 25 mM HEPES (pH 7.3), 1 ml per well. Kiwifruit preparations were examined at concentrations of 1.0, 3.0, and 10 mg/mL.

### Cyclosporin, Quercetin, and Dexamethasone Analysis

Splenocytes from OVA-sensitized mice (8 replicates) were utilized for the analysis of each compound tested, with the exception of Quercetin where the splenocytes derived from only 2 OVA-sensitized mice were examined. 5×10⁶ cells splenocytes from each mouse were plated out into the individual wells of a 24-well plate in complete RPMI-1640 medium containing 100 μg/mL OVA and 25 mM HEPES (pH 7.3), 1 ml per well. Cyclosporin was examined at concentrations of 0.0083, 0.083, and 4.15 μM. Dexamethasone was examined at concentrations of 0.01, 0.1 and 1 μM. Quercetin was examined at concentrations of 1, 10, and 25 μM. Wells treated with 1 μM dexamethasone, a potent glucocorticoid anti-inflammatory, served as positive experimental controls. Wells receiving only complete RPMI-1640 containing 100 μg/ml OVA and 25 mM HEPES (pH 7.3) served as OVA-only experimental controls. After 3 days of culture, the supernatants were collected and frozen. These supernatants were used to determine the levels of IL-13, and IFN-γ present in the various culture media, under the experimental conditions examined.

### Determination of Cytokine Levels in Culture Supernatants

The cytokine levels in the culture supernatants from all treatment and control wells were determined by ELISA assay. Two replicate ELISA plate wells were utilized for each cytokine level determination. As seen in Example 3 above only the expression of IL-13 and IFN-γ were analyzed for the purpose of estimating levels of activity present in the materials tested.

### Results

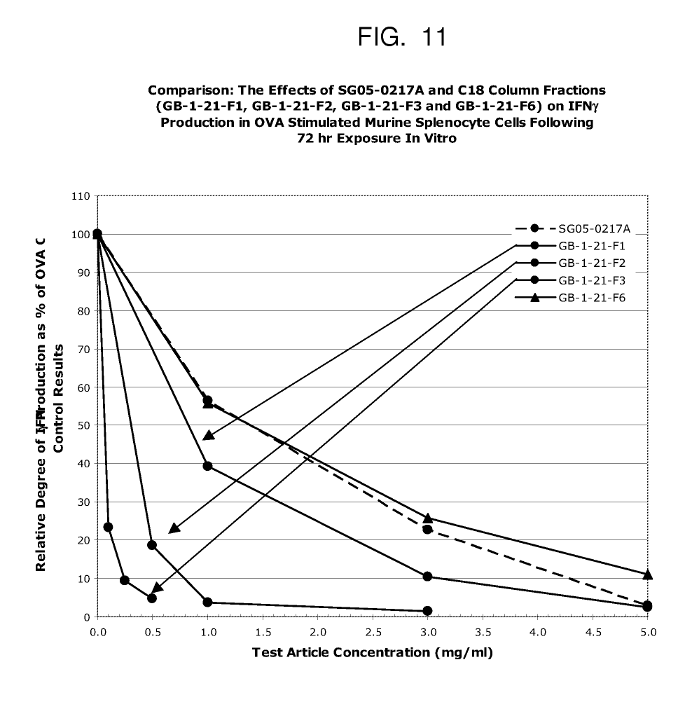

This experiment demonstrated a stronger suppression of the cytokines IL-13 and IFN-γ then observed from A. arguta preparations in this assay. Further confirmation of the ability of this chromatographic purification technique to extract activity from FD001 is demonstrated in FIG. 11. Compared to SG05-0217A the reference material for FD001, four of the five Cuts are substantially stronger suppressors of cytokines IL-13 and IFN-γ.

These data show that there are several viable alternative methods for preparing hardy kiwifruit preparations for therapeutic purposes.

### Example 5

The following example describes the results of a double-blind, placebo-controlled, outpatient study of the effectiveness of FD001 in adult subjects with atopic dermatitis of moderate severity.

The objective of this study was to obtain evidence of effectiveness of FD001 (PG102T), administered orally over a period of 42 days, in a small number of adult volunteers with atopic dermatitis (AD) of moderate severity. Secondary objectives of the study were to assess the tolerability and variability of response to FD001.

### Study Design

Subjects consumed FD001 powder (prepared as described in Example 1) in two 600 mg capsules (600 mg total dose of FD001) in the morning, or two capsules of placebo consisting of MCC, for a period of 42 days beginning on day 1 of the study. All but one study participant were on concomitant steroid therapy (oral or topical, various forms) when the study began. Subjects were instructed to halt use of steroid medications after day 14. Blood was drawn for routine biochemistry panel and hematology at four timepoints: on the screening visit for eligibility, and on days 1, 14 and 42 of the study. Levels of IgE and C-reactive protein were measured in blood on days 1, 14 and 42. Urine was collected on all four days for routine urinalysis. Efficacy assessments were done at each visit post screening. Subjects were either male or female, 19 to 65 years of age, and in generally good health. Subjects had active, atopic dermatitis of moderate severity defined by a Physician's Global Assessment (Feldman and Krueger, Annals of the Rheumatic Diseases, 64:ii65-ii68, 2005) score of three on the severity scale of 0 to 4. Subjects had AD involving a minimum of 10% of body surface area (BSA). Subjects were currently using a topical steroid for the treatment of AD and could not be nursing or pregnant. Safety and tolerability were assessed using adverse reaction reporting and standard blood chemistry, hematology and urinalysis.

### Statistical Methods

The primary efficacy variable was the change from baseline in the Physician's Global Assessment at day 42, with analysis using the Cochran Mantel-Haenszel test (Armitage et al., Statistical Methods in Medical Research, 4th Ed., Blackwell, Oxford, 2002). Secondary variables were the day 42 changes from baseline in the signs of AD (erythema, induration, oozing/crusting and pruritus severity scores), and in total BSA as analyzed using a two-sample t-test. Descriptive statistics were presented for all baseline and post-baseline study data by treatment group on days 1, 14, 28 and 42. These statistics included sample size, means, standard deviations, frequencies, percentages, and confidence intervals, as appropriate. Results from subject self-assessment questionnaires were tallied and presented by treatment group. Any adverse events occurring during the study were recorded. Descriptions of adverse events included the date of onset, the date the event ended or continued, the severity of the event, the attribution, action taken, therapy taken, and the outcome. These data were categorized by the number of subjects reporting adverse events, body system, severity, seriousness, and relationship to test article. Comparisons among treatment groups were made by tabulating the frequency of subjects with one or more adverse events classified into MedDRA terms (Medical Dictionary for Regulatory Activities. http://www.meddramsso.com) during the study.

Descriptive summary statistics for laboratory values and their associated change from baseline (day 1) were determined for all clinical laboratory assessments. Values outside the normal range were flagged in the data listings. In addition, “shift tables” were generated showing the number and percent of subjects that experienced changes in laboratory parameters during the course of the study (e.g., change from normal to high, based on the laboratory reference ranges). The following topical steroids are commonly used to treat the symptoms of atopic dermatitis. Those marked with ** were used by some individuals in this clinical trial:

++Betamethasone dipropionate (Diprolene), **Clobetasol 17-Propionate 0.05% (Dermovate), **Halobetasolpropionate (Ultravate), Halcinonide 0.1% (Halog), Amcinonide 0.1% (Cyclocort), Betamethasone dipropionate 0.5 mg (Diprolene, generics), Betamethasone valerate 0.05% (Betaderm, Celestoderm, Prevex), Desoximetasone 0.25% (Desoxi, Topicort), Diflucortolone valerate 0.1% (Nerisone), Fluocinonlone acetonide 0.25% (Derma, Fluoderm, Synalar), **Fluocinonide 0.05% (Lidemol, Lidex, Tyderm, Tiamol, Topsyn), Halcinonide (Halog), **Mometasone furoate 0.1% (Elocon), **Triamcinolone, Desonide 0.05% (Desowen), **Hydrocortisone valerate 0.2% (Westcort), Prednicarbate 0.1% (Dermatop) and Hydrocortisone 1.0% (Cortaid).

TABLE I

| | | number of | | % of | non- | % of |
|---|---|---|---|---|---|---|
| group* | | subjects | responders | total | responders | total |
| A | FD001 | 25 | 16 | 64.0 | 9 | 36.0 |
| | Placebo | 26 | 11 | 42.3 | 14 | 53.8 |
| B | FD001 | 18 | 11 | 61.1 | 7 | 38.9 |
| | Placebo | 24 | 11 | 45.8 | 13 | 54.2 |
| C | FD001 | 14 | 8 | 57.1 | 6 | 42.9 |
| | Placebo | 17 | 6 | 35.3 | 11 | 64.7 |

*A = all subjects B = clobestasol & ultravate users removed C = only users of HDRCRT-cortoid or triamcinolone

### Efficacy Results

An interim analysis conducted on the first 17 subjects to complete the study resulted in strong statistical trends in two secondary efficacy endpoints: erythema (p=0.13, ITT 17) and induration (p=0.09, ITT 17). At the time of the interim analysis, no statistical difference could be detected in the primary efficacy endpoints in this small sample size.

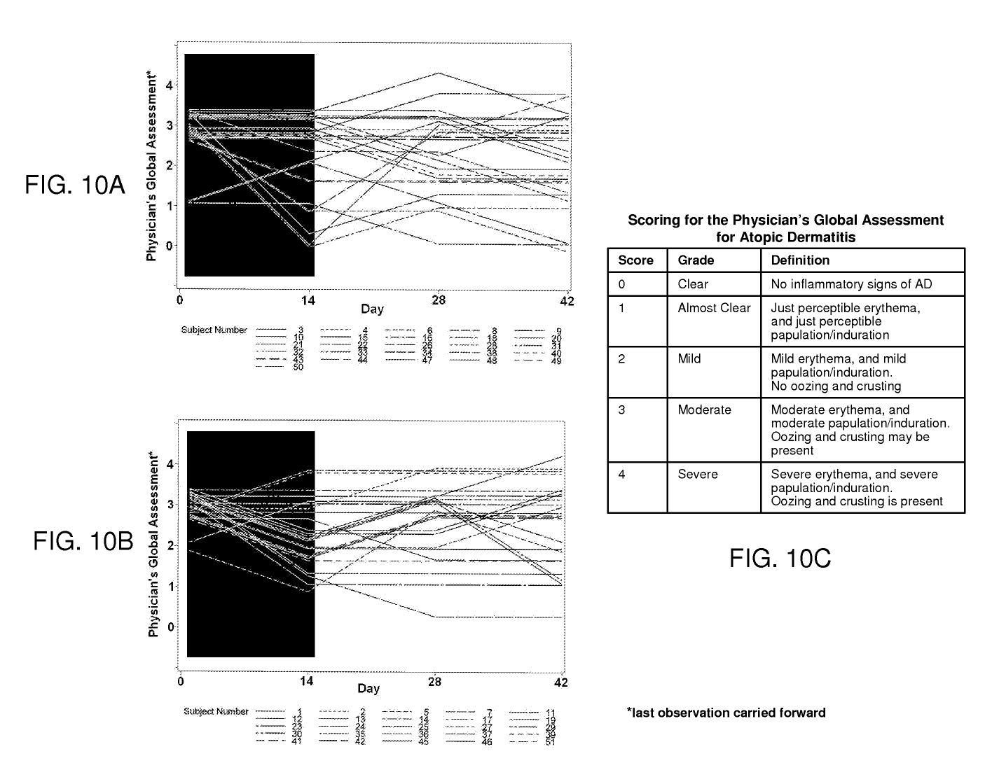

At the day 42 final analysis, there was no statistical significance demonstrated between treatment arms in the primary efficacy variable (Physician's Global Assessment) or the secondary efficacy variables (change from baseline in the signs of AD and percent change in BSA). At day 14, however, a significant distributional shift was observed in the primary efficacy endpoint. This shift, occurring between day 1 and 14, can be observed in FIGS. 10A and 10B for the responses of the placebo and test article recipients, respectively (PGA scoring criteria indicated). When subjects were sorted into responding and non-responding groups for the day 14 analysis (Table I, group A), a higher percentage of responders were observed in the FD001 treatment group, while non-responders represented a higher percentage of the placebo group. These results remained relatively consistent when subjects on more potent steroids (group B) were excluded, as well as for subjects only on moderate to mild steroid treatments (group C). Supplementary post hoc analyses revealed statistical significance (p=0.02) for the primary PGA endpoint and for the subject's self-assessment for redness (p=0.03) at day 14. In addition strong statistical trends were detected in self assessments at day 14 for itch, and for clinical signs assessments for oozing/crusting (p=0.08 and p=0.07, respectively). The observed efficacy of the treatment group compared to the placebo group at day 14 via the PGA endpoint dissipated by day 42.

These results indicate that use of FD001 as an adjunct to topical corticosteroid therapy is beneficial in the treatment of AD. Laboratory test results for days 1, 14 and 42 showed no safety related trends for any of the tests performed including clinical chemistry, complete blood count with differential, coagulation, indirect bilirubin, and urine macroscopy. No difference was found between treatment groups for IgE, C-reactive protein, or eosinophil counts.

### Safety Results

No serious adverse events were reported for either FD001 treatment or placebo. There were 12 non-serious events reported for FD001 and 13 for placebo. Of these, there were 10 mild to moderate and 2 severe events for FD001, and 13 mild to moderate events for placebo. None of the events were considered probable or related to the study test article or placebo.

### Conclusions

A strong distributional shift was noted in the primary endpoint (PGA) at day 14. For this reason, a post hoc analysis was conducted for this timepoint that revealed a statistically significant difference between treatment arms at the day 14 timepoint. In addition, some secondary endpoints demonstrated either statistically significant (erythema p=0.03 in the self assessment) or statistically strong trends (itch improvement p=0.08 in the self assessment and oozing/crusting p=0.07 in the signs of AD assessment). There were no serious adverse events reported in the trial. The number of adverse events reported in both the test article and placebo groups were comparable, and not attributable to the test article. There were no safety concerns detected in the clinical laboratory values. These data reaffirm preclinical small mammal and anecdotal human studies indicating that A. arguta fruit extracts are safe and well-tolerated by recipients, and are beneficial as an adjunctive therapy to steroid therapy.

### Example 6

The following example describes a randomized, double blind, placebo-controlled study to evaluate the use of a hardy kiwifruit extract to decrease the CADESI score (Olivry et al., Vet. Dermatol., 13:77-87, 2002; Hanifin et al., Exp. Dermatol., 10:11-18, 2001; Kunz et al., Dermatology, 1997, 195, 10-19) of atopic dogs.

The first objective of this study was to evaluate the efficacy of the A. arguta fruit extract (concentrate) FD001 as an adjunct therapy to a standard steroid treatment for atopic dermatitis (AD) in dogs. Response to treatment was assessed using the investigator's global evaluation which incorporates the CADESI scale and the owner's Pruritis assessments. A two week steroid-only lead-in period was applied to stabilize the dog and ensure an adequate steroid treatment response.

An additional objective of the study was to assess the efficacy of the kiwifruit extract as a monotherapy to decrease the need for steroid use in the management of AD clinical signs

A third objective of the study was to assess possible adverse effects of this treatment.

### Experimental Design and Methods

In order to test the proposed hypotheses a randomized, double blind, placebo controlled clinical study was completed.

A. Animals

Approximately 60 dogs with naturally occurring disease were selected.

Inclusion Criteria:
1. All dogs were judged healthy on physical examination excluding AD skin disease.
2. Diagnosis of non-seasonal AD (history for 1-6 yrs) of a mild to moderate severity, was based on suggestive history, compatible clinical signs, exclusion of other diseases, and at least 3 positive reactions in an intradermal skin test using the 57 most common allergens of the region.
3. Flea control treatment (monthly Advantage×1 month minimum)
4. Food trial to rule out possibility of a food component* or history of adequate food trial (within last 2 years)
5. If indicated, some dogs also underwent treatment for scabies.
6. All eligible dogs underwent the following withdrawal periods for concomitant medications:
   Two weeks for all antihistamines
   Two months for long acting injectable steroids (e.g. Depomedrol)
   Two weeks for topical steroids
   5-14 days for oral steroids (pruritus needed to be at its worse again)
   One month for oral CsA
   One month for all essential fatty acid supplements**
   No withdrawal for shampoos or antibiotics (but could not be used for study duration)
7. Animals were cleared of any secondary infections prior to enrollment.
8. A baseline minimum CADESI score of 25.
9. Dogs needed to continue the same diet for duration of study
10. Owners needed to sign Client consent prior to study start

Exclusion Criteria:
1. History of MRSA/MRSI
2. Animals with a pre-existing systemic disease (with the exception of controlled hypothyroidism)
3. Animals with exceptional severity of AD
4. Non responsive to steroid therapy during two week lead in period/or complete responders
5. Animals requiring medicated shampoos.
6. Animals requiring concurrent allergen specific immunotherapy unless treatment has stabilized disease and been ongoing for at least a year.

*The food trial was based on the specific dietary history of the dog, but introduced a novel source of protein and carbohydrate. Dogs were placed on an acceptable food trial diet (e.g. Venison and potato diet produced by Innovative Veterinary Diets) for 8 weeks. Pruritus and recurrence of infections were monitored during the trial. If those are observed during the trial, food allergy is ruled out. If they were absent or decreased, animals were rechallenged with previous diet and monitored for relapse of infection or Pruritus for up to 7 days. Therefore, other pruritic skin diseases that could potentially clinically look like AD will have been ruled out or controlled (like the flea allergy) prior to enrollment.

** Allowed 4 week withdrawal for cyclosporine and fatty acids.

B) Experimental Design

Selected dogs were randomized into 2 groups, test article or placebo at a ratio of 1:1. Therefore, approximately 30 dogs received test article while approximately 30 received placebo. The grouping was randomized and blinded to owners and investigators.

1. Test Article:

One treatment arm received KiwiBerry fruit extract/concentrate (Actinidia arguta FD001 preparation as described in Example 1) at a dose of 30 mg/kg once daily while the other treatment arm received placebo. Both the test article and placebo were formulated in a capsule that was administered to the dog by the owner. The capsule was allowed to be given as capsule or sprinkled over food

2. Steroid Treatment:

Both study groups were stabilized solely with a standard, low dose, oral steroid (Prednisolone) beginning on Study Day 1 until Study Day 14, at which time a reassessment was made to determine if the dog is still eligible to continue. Steroid dosing was applied as follows:
Days 1-3: 0.2 mg/kg BID
Days 4-14: 0.2 mg/kg every other day
   If dog meets continuing criteria at Day 14
Days 15-42 0.2 mg/kg every other day+assigned test article
   At the end of the first two weeks on steroid treatment the dogs were evaluated for steroid responsiveness and residual AD signs and symptoms (CADESI needs to be less than initial CADESI but >0).

3. Stage 1 Study Measures:

At the end of the two week lead in period the Investigator assessed the signs and symptoms of AD through the CADESI score and the owner's Pruritus diary. In order for the dog to continue to the 4 week adjunctive therapy period, the investigator's global evaluation was required to demonstrate improvement but not complete resolution in AD signs and symptoms as follows:
CADESI score reflects dogs are still lesional.
Pruritus diary shows improvement from Day 1 but not complete resolution

If the above criteria were met, the dog was placed on combination therapy and continued receiving every other day Prednisolone dosing at 0.2 mg/kg with the DAILY assigned test article. The dog owner was supplied with the oral steroid for the remaining study period in Stage 1 (4 weeks).

If the dog had not responded to the standard steroid treatment, study participation was discontinued and another dog was selected until target enrollment was reached.

During the 6 weeks of treatment (two week steroid lead in period and four week combination therapy), dogs were assessed via the CADESI scale and Pruritus diaries at the intervals noted in the Schedule of Events. At the end of the 6 week treatment period, the dogs in each group were compared in terms of CADESI scores, clinical symptoms and the Pruritus diary kept by the owner. The dogs that demonstrated improvement in their CADESI scores and were deemed by the investigator as a good candidate, were eligible for Stage 2 (Kiwiberry monotherapy) of this protocol.

Blood specimens were collected to assess safety and secondary endpoints. Labs for CBC, Chemistry, Total and allergen specific IgE levels, and TARC were conducted at the time points specified in the Schedule of Events. CBC, chem. Panel were run immediately (at Colorado State University). Total, allergen specific IgE and TARC samples were stored −70° C.

4. Stage Two Study Measures:

Dogs that advance to Stage 2 began a four week, open label, monotherapy period. Placebo and test substance dogs from stage were allowed to enroll, as blinding was not broken until after stage two was completed. If a dog experienced a relapse in their AD signs or required rescue meds prior to the end of the four week period, they were considered to be Stage Two treatment failures and discontinued from the study. Dogs that maintained their improvement in Stage Two were considered Stage Two treatment responders.

Blood specimens were collected to assess safety and secondary endpoints. Labs for CBC, Chemistry, Total and allergen specific IgE levels, and TARC were conducted at the time points specified in the Schedule of Events.

Study measurements in Stage 1 were taken at study visits as indicated below.

Schedule of Study Events- Stage One

| | | Day | Day | Day | Day | Day |
|---|---|---|---|---|---|---|
| Study Procedures | Screen | 1 | 14 | 28 | 35 | 42 |
| Inc/Exc | X | | | | | |
| Eval of Signs/CADESI | | X | X | X | X | X |
| Cytology for lesional | | X | X | X | X | X |
| areas as needed | | | | | | |
| Pictures | | X | X | X | X | X |
| Labs: CBC, Chemistry | | X | X | | | X |
| Steroid treatment | | X | X | X | X | X |
| dosing [symbol] | | | | | | |
| Study Dosing [symbol] | | | X | X | X | X |
| Weekly (owner) Pruritus | | X | X | X | X | X |
| diary | | | | | | |
| Steroid responsiveness | | | X | | | |
| assessment | | | | | | |

[symbol]

Steroid treatment dosing began at Day 1 and continued through Day 42 [symbol]
Test article dosing began at Day 14 and is once every day from Day 14 through Day 42
Weekly Pruritus diary completed every week from Day 1 through Day 42
Rechecks had to be scheduled within +/− ONE day
Patients had to be seen by same clinician or both clinicians have to have performed CADESI's at prior visits
The period between screening and enrollment into the study (Day 1) did not exceed 3 weeks and was a minimum of 1 day if all inclusion criteria noted above were satisfied, (food trial had to be performed prior to screening day).

Schedule of Study Events- Stage Two

| | | Day | Day | Day | Day | Day |
|---|---|---|---|---|---|---|
| Study Procedures | Screen | 1 | 14 | 28 | 35 | 42 |
| Inc/Exc | X | | | | | |
| Eval of Signs/CADESI | | X | X | X | X | X |
| Cytology for lesional | | X | X | X | X | X |
| areas as needed | | | | | | |
| Pictures | | X | X | X | X | X |
| Labs: CBC, Chemistry, | | X | X | | | X |
| Steroid treatment | | X | X | X | X | X |
| dosing [symbol] | | | | | | |
| Study Dosing [symbol] | | | X | X | X | X |
| Weekly (owner) Pruritus | | X | X | X | X | X |
| diary | | | | | | |
| Steroid responsiveness | | | X | | | |
| assessment | | | | | | |

[symbol]

Test article dosing began as monotherapy beginning Day 1 and was once every day through Day 28
Weekly Pruritus diary completed every week from Day 1 through Day 28

5. Evaluation of Efficacy by Owners

A score for Pruritus (scale from 0 to 5, with higher numbers meaning more severe Pruritus) was assigned by owners weekly (Table 2).

TABLE 2
Criteria for the evaluation and scoring of Pruritus by the owner.

| Score | Definition |
|---|---|
| 1 | Mild (scratching, rubbing, chewing or licking, <10% of time |
| | observed) |
| 2 | Mild-moderate (scratching, rubbing, chewing or licking, 10-30% |
| | of time observed) |
| 3 | Moderate (scratching, rubbing, chewing or licking, 30-50% of |
| | time observed) |
| 4 | Moderate-severe (scratching, rubbing, chewing, licking, 50-75% |
| | of time observed, still able to relax/sleep at nigh) |
| 5 | Severe (scratching, rubbing, chewing or licking all the time, |
| | even at night and during a meal) |

6. Clinical Evaluation of Efficacy by Investigator

Erythema, lichenification and excoriations were evaluated and scored by the investigator on Days 1, 14, 28, 35 and 42 of each treatment period using the Canine Atopic Dermatitis Extent and Severity Index Score (CADESI, Table 3). This scoring system was used previously to assess severity of AD in dogs (Olivry et al. Vet. Dermatol 2000; 11: 47) and was adapted from scoring systems used for human AD (Hanifin et al., Exp Dermatol 2001; 10 (1): 11-8; Kunz et al., Dermatology 1997; 195 (1): 10-9). A scale from 0 to 3 was used for each parameter (0=absent, 1=mild, 2=moderate, 3=severe). Thus for each area evaluated the sum of the three scores ranged from 0 to 9. The total score ranged from 0 to 360. The primary endpoint in this study was the investigator's overall evaluation using the CADESI score and the Pruritus diary. Treatment failure was defined as follows: Return to baseline (Day 1) CADESI score or higher when either related to worsening of AD or outbreak of secondary infection. Cytology of lesional areas was conducted at each CADESI assessment (weekly). Subjects that did not show an improvement in CADESI score were considered a treatment failure.

Dogs were withdrawn from the study if any of the following were met:
Owner was non-compliant with protocol
Subject developed a secondary infection requiring treatment
Pruritus was severe enough in localized areas that self-mutilation occurs.

Secondary efficacy endpoints included the Pruritus ratings, and Safety labs.

Dogs whose CADESI score improved and that did not require rescue medications during treatment weeks 2-6 (adjunctive therapy period) were deemed responders and were eligible for Stage 2 of this protocol, while the dogs that required rescue meds were dropped from the study and assessed as treatment failures or non-responders. Other than the test article and the standard steroid supplied by the investigator, no additional medications, anti-histamines, for example, were allowed during the treatment period unless the dog was categorized as a treatment failure.

7. Blood Samples

On days 1, 14 and 42 of the treatment period, blood was collected for CBC, Chemistry, Total and allergen specific IgE levels, and TARC.

8. Safety Variables

Safety was evaluated on the basis of incidence of adverse effects and laboratory profile. Samples for complete blood cell counts (CBC w/differential) aPTT, and chemistry panel profiles were drawn on days 1, 14 and 42.

9. Statistics

Data was analyzed by a Chi Square test for the primary endpoint and ANOVA analysis for secondary endpoints, (e.g CADESI, Pruritus rating). Additional detail on the analysis plan can be found in the Statistical Analysis Plan document for this protocol. An interim analysis was conducted when the first half of the target enrollment has completed the study. A p<0.05 was considered significant.

### Summary and Results: Overview

This study provides evidence of EFF1001 test article activity in the population observed and suggests that EFF1001 is a useful adjunctive and potential monotherapy product in the management and maintenance of canine AD signs and symptoms as well as for the sparing of steroid use. In this study, the majority of dogs who remained steroid free and maintained or improved on their AD status during Stage 2 were recipients of the combination treatment (i.e. low dose steroid and EFF1001) in Stage 1.

### Summary of Canine Study Stage 1

The study enrolled 77 dogs across 4 veterinary clinics in the United States. Dogs enrolled in this study were diagnosed with non-seasonal Atopic Dermatitis for 1-6 years of mild to moderate severity. Stage 1 was a six week treatment period which included a two week, low dose (0.2 mg/kg) steroid only induction to stabilize the atopic dermatitis. At Day 14, the study introduced 30 mg/kg of EFF1001 or placebo in conjunction with the low dose steroid (q EOD steroid dosing) for the remaining 4 weeks of Stage 1. Frequent clinic visits (i.e. approximately every week) ensured that CADESI and Pruritus measurements continued in a consistent fashion throughout the study.

Seventy-one percent (55/77) of the dogs enrolled completed Stage 1. An improvement in Pruritus (a partial primary endpoint) was observed in those who received EFF1001 (p=0.0594). At Day 42 in Stage 1, the CADESI scores in the EFF1009 group improved more than the Placebo Group (−6.9 vs. −4.0), although the between-group difference is not statistically significant (p=0.4371).

Sixty-four percent of the dogs completing Stage 1 (35/55) were advanced to Stage 2; a 4 week, EFF1001 only (monotherapy) treatment phase. Approximately equal populations of placebo and test article dogs advanced from Stage 1 to Stage 2 based solely upon their suitability as determined by the investigators who remained blinded to their treatment assignment. The investigators relied heavily upon the dog's CADESI scores, pruritis scores, overall condition and the owners' assessments.

### Safety Results

One serious adverse event was reported on study for a dog at site #2 but was unrelated to the test article. Dog #140 was hospitalized and died subsequent to injuries sustained in a dogfight with two other dogs. No other serious or non-serious adverse events were reported.

### Summary of Canine Study Stage 2

35 dogs (18 EFF1001 recipients vs. 17 Placebo recipients from Stage 1) entered into Stage 2, a monotherapy treatment period with all dogs receiving only EFF1001. A significant advantage for the EFF1001 recipients from Stage 1 was observed in that they were 3.5 times more likely to sustain the benefit from the therapy in Stage 2 (RR=3.54; OR=16.26, p=0.0006, Fisher's exact test) than their Stage 1 Placebo recipient counterparts. Of the 35 dogs entering into Stage 2, 19 were able to maintain or improve their status in relation to CADESI and Pruritus scores, 15 of these (or 79%) had been EFF1001 recipients in Stage 1. Alternately, most of those who were Placebo recipients in Stage 1 experienced a relapse in their AD signs in Stage 2 and required rescue medications (81% vs. 19% in the EFF1001 group). This resulted in a statistically significant difference in the number of responders in Stage 2 (5.9% in Placebo recipients from Stage 1 vs. 66.7% in those who received EFF1001 from Stage 1) with a p-value of 0.0002. This result indicates that after four weeks of adjunctive treatment with EFF1001 and low dose steroid (Stage 1), subjects experienced longer periods of disease stability and steroid sparing when compared to dogs first treated with steroid and placebo (Stage 1).

CADESI mean score in the Stage 1 Placebo recipient group dipped at the start of Stage 2 because Stage 1 dogs whose condition had deteriorated were not advanced to Stage 2. By Day 7 of Stage 2 it was evident that most of these Stage 1 Placebo dogs had symptom flaring, based on increased CADESI. In contrast, most Stage 1 EFF1001 group dogs did not relapse during Stage 2. When using start of Stage 2 as baseline, the relapse phenomenon in Placebo group CADESI scores at Day 21 of Stage 2 is statistically significant compared to the treatment group (11.7 placebo vs. 1.8 in EFF1001, p=0.046) and at Day 28 (14.4 vs. 0.8, respectively p=0.02) with missing value imputation (Table 13.2).

Dogs in the EFF1001 group who had already experienced significant improvement in Pruritus scores over Placebo (−0.4 vs. +0.1 in Placebo, p=0.06) in Stage 1, continued to sustain the benefits from the therapy through Stage 2, whereas the Placebo group worsened (−0.1 vs. +0.8 in Placebo). (Table 12.1). When using the start of Stage 2 as baseline, the Placebo group Pruritus scores worsened relative to the treatment group at both Day 21 (0.4 placebo vs. −0.1 in EFF1001 recipients) and at Day 28 (0.5 vs. −0.2, respectively) with missing value imputation (Table 13.2).

### Continuation of Treatment Under Compassionate Use

Thirteen out of the 19 dogs (or 64%) that completed Stage 2 had owners who requested repeat, monotherapy treatments after the study period was over. Seventy-seven percent (77%) of these dogs were EFF1001 recipients in Stage 1. Compassionate use test article was supplied in six week intervals and the completion of Pruritus diaries was requested. To date, the longest running use of EFF1001 that is documented for a study dog (#127, site #2) is over one year and continues to appear helpful in the management of this dog's AD symptoms. This data strongly suggests that duration of exposure and early adjunctive treatment is a key component of EFF1001's subsequent efficacy in the management of AD signs and symptoms and its ability to aid in steroid sparing.

### Conclusions

The efficacy of the combination of steroid therapy and treatment with a hardy kiwifruit preparation of the invention has been demonstrated by showing a surprising superior and beneficial effect of the combination therapy in treating dogs with AD as compared to results observed with either therapy alone. Further, the kiwifruit preparation becomes an effective monotherapy when used following the use of the kiwifruit preparation and steroid combination. Without being bound by theory, the present inventors believe that the hardy kiwifruit preparation is synergizing with the steroid to provide a superior therapeutic result in the treatment of AD. Accordingly, FD001 and other kiwifruit preparations as described herein, when administered concomitantly with a steroid-based therapy or when used as a monotherapy following the concomitant use with a steroid-based therapy, enhances the therapeutic benefits to dogs with AD and as an added benefit, decrease the need for steroid use in the management of AD in dogs. The ability to use smaller amounts of steroid and/or less frequent steroid administration has the significant advantage of reducing the side effects that occur with steroid use. The extract can be administered as a capsule, a powder mixed with food, or as a component of the food. Dietary supplementation with kiwifruit preparations is effective in supporting healthy skin in dogs with atopy when used alone or in combination with low dose steroids.

One manifestation of allergy, atopic dermatitis, is a common skin disorder in children and is usually observed during the first 6 months of life (Spergel and Paller, J. Allergy Clin. Immunol., 112:S128-S139, 2003). The prevalence of AD appears to be increasing worldwide, as are other atopic disorders, including asthma (Larsen and Hanikin, Immunology and Allergy Clinics of North America, 22:1-25, 2002; Wollenberg et al., Clin. Exp. Dermatol., 25:530-534, 2000; Mannino et al., Mor Mortal Wkly Rep CDC Surveill Summ., 47:1-27, 1998; Linneberg et al., Allergy, 55:767-772, 2000). AD patients experience serious negative effects on the quality of life, and currently available treatments can be both a source of adverse side-effects, and a financial burden for both the family and society. The cutaneous manifestations of atopy often represent the beginning of the atopic march. On the basis of several longitudinal studies, approximately half of AD patients will develop asthma, particularly with severe AD, and two thirds will develop allergic rhinitis (Leung et al., J. Clin. Invest., 113:651-657, 2004; Spergel et al., J. Clin. Invest., 101:1614-1622, 1998). Identification of a safe and effective treatment for AD would be greatly welcomed. Efficacy of FD001 may also decrease the amount or potency of steroids or other medications used in the management of AD, asthma, or allergic rhinitis.

As the subject of later trials, the invention contemplates the use of the hardy kiwifruit preparations described herein, for adjunctive treatment with steroid therapy, which may be followed by hardy kiwifruit monotherapy for AD, asthma, allergic rhinitis, or other leukotriene-mediated conditions such as food allergies and chronic urticaria. As a logical extension of this work, the use of kiwifruit preparations in combination with steroid therapy as treatment for allergic conditions in any small mammals (e.g. dogs, see also Example 7) may be explored further.

Each reference or publication described herein is incorporated herein by reference in its entirety.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to regulate an immune response in a mammal, comprising administering at least one hardy kiwifruit preparation and at least one steroid to the mammal in an amount sufficient to regulate an immune response in the mammal.

2. The method of claim 1, wherein the hardy kiwifruit is selected from the group consisting of: Actinidia arguta, Actinidia kolomikta and Actinidia polygama.

3. The method of claim 1, wherein the hardy kiwifruit is Actinidia arguta.

4. The method of claim 1, wherein the hardy kiwifruit preparation is an extract of the hardy kiwifruit.

5. The method of claim 1, wherein the hardy kiwifruit preparation is produced by extraction in distilled water.

6. The method of claim 1, wherein the hardy kiwifruit preparation is produced by extraction in a non-polar solvent.

7. The method of claim 1, wherein the hardy kiwifruit preparation is produced by chromatographic purification of an aqueous extract.

8. The method of claim 7, wherein the chromatographic purification involves the use of a reverse phase chromatographic media.

9. The method of claim 7, wherein the chromatographic purification involves the use of a normal phase chromatographic media.

10. The method of claim 1, wherein the steroid comprises a naturally occurring steroid.

11. The method of claim 1, wherein the steroid comprises a synthetic steroid.

12. The method of claim 1, wherein the steroid comprises a steroid selected from the group consisting of: animal steroids, plant steroids and fungal steroids.

13. The method of claim 1, wherein the steroid comprises a corticosteroid.

14. The method of claim 1, wherein the mammal has or is at risk of developing a condition in which enhancement of a Th1 response and/or suppression of a Th2 response is desirable.

15. The method of claim 1, wherein the mammal has or is at risk of developing an allergic disease or non-allergic inflammatory disease.

16. The method of claim 15, wherein the allergic disease is regulated by leukotrienes.

17. The method of claim 15, wherein the allergic disease is selected from the group consisting of: atopic dermatitis, asthma, food allergy, allergic rhinitis, and chronic urticaria.

18. The method of claim 15, wherein the allergic disease is atopic dermatitis.

19. The method of claim 1, wherein the step of administering comprises administering one or both of the hardy kiwifruit preparation and the steroid with a carrier, adjuvant, or excipient to the mammal.

20. The method of claim 1, wherein the step of administering comprises providing one or both of the hardy kiwifruit preparation and the steroid to the mammal as a tablet, a powder, an effervescent tablet, an effervescent powder, a capsule, a liquid, a suspension, a granule or a syrup.

21. The method of claim 1, wherein the step of administering comprises providing one or both of the hardy kiwifruit preparation and the steroid to the mammal in one or more health foods.

22. The method of claim 1, wherein the step of administering comprises administering one or both of the hardy kiwifruit preparation and the steroid as a topical composition.

23. The method of claim 1, wherein the step of administering comprises providing one or both of the hardy kiwifruit preparation and the steroid to the mammal in a feed or feed ingredient.

24. The method of claim 1, wherein the hardy kiwifruit preparation is formulated in the same composition with the steroid.

25. The method of claim 1, wherein the hardy kiwifruit preparation is formulated in a different composition than the steroid, but administered contemporaneously with the steroid.

26. The method of claim 1, wherein, following a period of administration of the hardy kiwifruit preparation and the steroid, the mammal is administered with the hardy kiwifruit preparation in the absence of the steroid.

27. The method of claim 26, wherein the period of administration of the hardy kiwifruit preparation and the steroid to a mammal prior to administration of the hardy kiwifruit preparation alone is from one day to several years.

28. The method of claim 26, wherein the period of administration of the hardy kiwifruit preparation to a mammal alone following the administration of the hardy kiwifruit preparation and the steroid is from one day up to several years.

29. A method to reduce at least one symptom of inflammation in a mammal, comprising administering a hardy kiwifruit preparation and at least one steroid to the mammal in an amount sufficient to reduce said symptom of inflammation.

30. The method of claim 29, wherein symptom of inflammation is selected from the group consisting of: itching, redness, oozing and crusting, thickening, hair loss, and swelling of the skin.

31. A composition for regulating an immune response in a mammal or for reducing at least one symptom of inflammation in the mammal, the composition comprising at least one hardy kiwifruit preparation and at least one steroid.

32. The composition of claim 31, wherein the composition is selected from the group consisting of: a pharmaceutical composition, a health food, a food ingredient, an animal feed, and a cosmetic composition.

33. The composition of claim 31, wherein the hardy kiwifruit is selected from the group consisting of: Actinidia arguta, Actinidia kolomikta and Actinidia polygama.

34. The composition of claim 31, wherein the hardy kiwifruit is Actinidia arguta.

35. The composition of claim 31, wherein the hardy kiwifruit preparation is an extract of the hardy kiwifruit.

36. The composition of claim 31, wherein the hardy kiwifruit preparation is produced by extraction in distilled water.

37. The composition of claim 31, wherein the hardy kiwifruit preparation is produced by extraction in a non-polar solvent.

38. The composition of claim 31, wherein the hardy kiwifruit preparation is produced by chromatographic purification of an aqueous extract.

39. The composition of claim 38, wherein the chromatographic purification involves the use of a reverse phase chromatographic media.

40. The composition of claim 38, wherein the chromatographic purification involves the use of a normal phase chromatographic media.

41. The composition of claim 31, wherein the steroid comprises a naturally occurring steroid.

42. The composition of claim 31, wherein the steroid comprises a synthetic steroid.

43. The composition of claim 31, wherein the steroid comprises a steroid selected from the group consisting of: animal steroids, plant steroids and fungal steroids.

44. The composition of claim 31, wherein the steroid comprises a corticosteroid.

45. The composition of claim 31, wherein the composition is formulated for oral administration.

46. The composition of claim 31, wherein the composition is formulated for topical administration.

47. An animal feed product, comprising the composition of claim 31.

48. The animal feed product of claim 47, wherein the animal feed product is selected from the group consisting of: a pet food, a livestock food, a pet treat, a pet chew, and a supplement.
